(19) United States
(12) Patent Application Publication
(10) Pub. No.: US 2007/0224191 A1
(43) Pub. Date: Sep. 27, 2007

(54) Methods of treatment of Ulcerative Colitis and Crohn's disease with anti-CD3 antibodies

(75) Inventors: Ian Walters, Swampscott, MA (US); James Lowder, Pleasanton, CA (US)

Correspondence Address: TOWNSEND AND TOWNSEND AND CREW LLP, TWO EMBARCADERO CENTER, EIGHTH FLOOR, SAN FRANCISCO, CA, 94111-3834, US

(73) Assignee: PDL BioPharma, Inc., Fremont, CA (US)

(21) Appl. No.: 11/713,465

(22) Filed: Mar. 1, 2007

Related U.S. Application Data
(63) Continuation-in-part of application No. 10/729,795, filed on Dec. 5, 2003.
(60) Provisional application No. 60/450,183, filed on Feb. 25, 2003.
(60) Provisional application No. 60/431,649, filed on Dec. 5, 2002.

(51) Int. Cl. A61K 39/395

(52) U.S. Cl. 424/133.1; 424/130.1

(57) ABSTRACT

The present invention provides a method of treating autoimmune diseases. In particular, it provides a method for the treatment of ulcerative colitis or Crohn's disease comprising administering to a subject a therapeutically effective amount of a pharmaceutical formulation comprising an antibody, wherein said antibody binds to CD3.

### CROSS-REFERENCES TO RELATED APPLICATIONS

The present application is a continuation in part of 10/729,795 filed Dec. 5, 2003, which is a non-provisional application claiming priority to provisional application Ser. Nos. 60/431,649, filed Dec. 5, 2002, and 60/450,183, filed Feb. 25, 2003.

### FIELD OF THE INVENTION

The present invention applies the technical fields of immunology and the treatment of autoimmune disease. In particular, it concerns methods of treating Ulcerative Colitis and Crohn's disease with anti-CD3 antibodies.

### BACKGROUND OF THE INVENTION

Inflammatory bowel disease (IBD), including ulcerative colitis (UC) and Crohn's disease are chronic, inflammatory diseases of the small and large intestine. It is estimated that approximately 1 million Americans suffer from IBD, about half of them with UC. The exact cause of UC and Crohn's disease is not known, but IBD is generally regarded as a chronic inflammatory disease.

The major symptoms of ulcerative colitis are bloody diarrhea and abdominal pain, often with fever and weight loss. The clinical course of ulcerative colitis is variable. The majority of patients will suffer a relapse within a year of the first attack. There may, however, be prolonged periods of remission with only minimal symptoms. Some patients may have mild to moderate disease of an intermittent nature and can be managed without hospitalization. In approximately 15 percent patients, the disease assumes a more fulminant course, involves the entire colon, and present with severe bloody diarrhea and systemic signs and symptoms. The patients are at risk to develop toxic dilation and perforation of the colon and represent a medical emergency (Harrison's Principles of Internal Medicine ₁₂th Edition, McGraw-Hill Inc. (1991)).

Currently, there is no medical cure for UC. The available treatments aim at reducing inflammation of the epithelium of the colon, thereby controlling gastrointestinal (GI) symptoms. The major classes of medications used today include aminosalicylates, corticosteroids (eg, prednisone), and immunomodulatory medicines (eg, azathioprine and cyclosporine). Colectomy will eliminate the disease; however, this surgical procedure is potentially compromised by pouchitis, pouch dysfunction, or dysplasia (Miner P B., et al., In Kirsner J B, ed. Inflammatory Bowel Disease. 5th ed. Baltimore: Williams and Wilkins: 299-304 (2000)).

The first attack of UC is usually mild with a 91 % rate of remission using standard medical therapy alone. However, more than 70% of patients will experience relapse that follows a chronic intermittent or chronic continuous course. Patients will usually be treated initially with a combination of steroids with or without an oral (PO) 5-amino-salicylate agent. If the disease fails to respond to PO steroids, IV steroids or 6 mercaptopurine can be added. For patients whose disease does not respond to these therapies, a limited repertoire of agents, including a short course of cyclosporine or investigational agents, is available. Cyclosporine is successful in inducing remission in approximately 50% of patients. However, cyclosporine is associated with a high level of acute toxicity, and up to 70% of cyclosporine-treated patients will require surgery within one year to control their disease (Naftali T, et al., Isr Med Assoc J; 2(8): 607-609 (2000); Haslam N, et al., Eur J Gastroenterol. Hepatol. 12(6): 657-660 (2000); Rowe F A, et al. Am. J Gastroenterol; 95(8): 2000-2008 (2000)). This population represents a significant proportion (29%) of UC patients, and there is substantial morbidity associated with surgical intervention (Singleton J W, et al., In Kirsner J B and Shorter R G, eds. Inflammatory Bowel Disease. ₄th ed. Baltimore: Williams and Wilkins; 335-343 (1995)).

The ineffectiveness of these existing treatment approaches is at least partly due to their disease control mechanism, such as the non-specific immunosuppression rather than the specific modulation of activated T-cells. These therapeutic agents only cause temporary decrease in the activation and proliferation of all T-cells. As a result, the symptoms of the patients come back right away when the treatment stops.

In view of the deficiency the existing methods of treating ulcerative colitis, it is desirable to develop more effective therapeutic agents, especially for the type of UC that does not respond to conventional nonspecific immunosuppression and thereby has a poor prognosis.

The CD3 complex on T-cells is closely associated with the T-cell receptor (TCR) heterodimer and plays important role in T-cell activation upon antigen binding. It is believed that T lymphocytes are the primary immune cell mediating IBD induction and progression. Because UC has components of both Th1 and Th2 T cell inflammatory mediators associated with its disease pathology, it is proposed that an antibody that recognizes both Th1 and Th2 cells, such as the anti-CD3 antibody, could provide therapeutic benefit in UC (Elson C., et al., In Kirsner J B, ed. Inflammatory Bowel Disease. ₅th ed. Baltimore: Williams and Wilkins: 208-239. (2000)). Unlike the traditional therapeutic agents, such as cyclosporine, anti-CD3 antibodies only inhibit the proliferation or induce apoptosis of the activated T-cells without disturbing the function of the other T-cells. Thus, anti-CD3 antibodies are more selective and should have an impact on disease activity long after the termination of the treatment.

Pharmacological and toxicological testing indicated that anti-CD3 antibodies such as visilizumab, were well-tolerated in chimpanzees (Investigator's Brochure, Visilizumab (Nuvion®; HuM291): Autoimmune and Inflammatory Diseases. Edition No. 3; PDL, Inc. April, (2002)). This antibody was also proved to be well-tolerated and offer desired clinical efficacy in Phase I and II clinical studies for the treatment of acute graft-versus-host disease (GVHD) (Carpenter Pa., Appelbaum FR, Corey L, et al., Blood 99(8): 2712-2719 (2002)) and psoriasis (pending U.S. Patent application Ser. No.: 10/001,234, filed Oct. 30, 2001). It is also reported that treatment with another anti-CD3 antibody, HuOKT3γ1 (Ala-Ala), reversed acute renal allograft rejections in a phase I clinical trial study (Woodle, E. S., et al., Transplantation Vol. 68: 608-616 (1999)). In addition, this antibody mitigated the deterioration in insulin production and improved metabolic control during the first year of type 1 diabetics mellitus (Herold, K. C., et al., N. Engl. J. Med. Vol. 346: 1692-1698 (2002)). A phase I/II clinical trial study demonstrated the clinical efficacy for the treatment of psoriasis with anti-CD3 antibodies (America College of Rheumatology Meeting November, 2002).

However, no clinical studies have been conducted to examine the possibility of treating ulcerative colitis with anti-CD3 antibodies. The present invention discloses the Phase I/II clinical studies for the treatment of ulcerative colitis with anti-CD3 antibodies in and provides for methods of using anti-CD3 antibodies for the treatment of UC, preferably, the severe steroid-refractory ulcerative colitis. The methods of the present invention offer superior clinical efficacy and long-lasting beneficial results compared to the existing treatment approaches.

### BRIEF SUMMARY OF THE INVENTION

The present invention provides for a method for the treatment of diseases of the immune system, such as autoimmune diseases.

The present invention provides for a method of treating ulcerative colitis in a patient in need of such a treatment comprising administering to said patient a molecule that specifically modulates activated T-cells, preferably inhibits proliferation or activation of T-cells.

The present invention provides for a method of treating ulcerative colitis in a patient in need of such a treatment comprising administering to said patient a therapeutically effective amount of a pharmaceutical formulation comprising an antibody, wherein said antibody binds to CD3. Said treatment causes a reduction in the symptoms of the disease, such as clinical or/and endoscopic remission of the disease as measured, e.g., by the Modified Truelove and Witts Severity Index (MTWSI) score (see Table 4) of said patient. Preferably, the antibody is neutralizing, i.e., neutralizes one or more or all biological activities of CD3. Preferably, the antibody is the mouse M291 antibody (see U.S. Pat. No. 5,834,597) or an antibody that recognizes the same epitope as the mouse M291 antibody. Preferably, the antibody is a humanized or human antibody. Most preferably, the antibody is visilizumab (see U.S. Pat. No. 5,834,597) or an antibody that recognizes the same epitope as visilizumab.

The present invention provides methods of treating Crohn's disease or ulcerative colitis by administering an anti-CD3 antibody at a dose of 1-15 μg/kg patient body weight. In other the dose is 1-10 μg/kg. In some methods the dose is 1-5, 5-10, 5-15 or 10-15 μg/kg. In some methods the dose is 4-6 μg/kg. In some methods the dose is 5 μg/kg. In some methods the dose is 8-12 μg/kg. In some methods the dose is 10 μg/kg. In some methods, the dose is 2.5, 2.5-5, 2.5-7.5 or 2.5-10 μg/kg. In some methods, a single dose is administered per relapse of the disease. Optionally, two and only two doses are administered per relapse of the disease. The two doses can be administered on consecutive days. The method is particularly suited to a patient's whose disease is refractory to treatment with an antibody to TNF-alpha and/or intravenous corticosteroids. The method can be used for patients having Crohn's disease particularly when the disease is refractory to treatment an antibody to TNF-alpha. The method can be used for luminal Crohn's disease. Some methods involve also administering a corticosteroid before administration of the anti-CD3 antibody.

In some methods the antibody used in the treatment is a humanized antibody, such as a humanized M291 antibody. In some methods the humanized M291 antibody is visilizumab. In some methods the antibody binds to the same epitope as visilizumab. In some methods the antibody has an amino acid sequence that is at least 80% identical to the amino acid sequence of visilizumab. In some methods the antibody has CDR regions that have amino acid sequences that are identical to the amino acid sequences of the CDR regions of visilizumab. In some methods the antibody has the same constant region as visilizumab. The method can be used for patients having an Epstein Barr viral titer of greater than 5000 copies per ml before administering the antibody.

### BRIEF DESCRIPTION OF THE DRAWINGS

FIG. 1 depicts the CD3 and CD4 counts (cells/μL) of patients. The arrow indicates the day at which visilizumab is administered to the patient (day 0).

FIG. 2 depicts the EBV DNA copies (/μL) measured from patients treated with visilizumab. Visilizumab was administered to the patient in day 1.

FIG. 3 depicts the clinical response (based on the MTWSI score) to the treatment with visilizumab. The number of patients treated was eight and the visilizumab dose was 15 μg/Kg administered on days 1 and 2 by intravenous infusion.

FIG. 4A depicts the endoscopic appearance of “severe” mucosal changes. These changes resolved to “normal” colon in 30 days after treatment with 2 doses of 15 μg/Kg of visilizumab on days 1 and 2. FIG. 4B depicts the endoscopic appearance of a patient who achieved a complete response in 30 days.

FIGS. 5A-5B depict the H&E stained biopsies taken during the endoscopies described in FIGS. 4A-4B. FIG. 5A depicts an ulcer where the epithelial cells are entirely lost. The submucosal remaining is densely infiltrated with granulocytes. These granulocytes leaking into the colonic lumen represent the pus seen in FIG. 4A. FIG. 5B is a H&E photomicrograph showing essentially normal colonic mucosa with no edema, and no granulocytes or lymphocytes infiltrating the submucosa.

FIG. 6 depicts the clinical response at day 30 for treatment of ulcerative colitis with a range of visilizumab doses.

FIG. 7 depicts the remission rate at day 30 for treatment of ulcerative colitis with a range of visilizumab doses.

FIG. 8 depicts the mucosal healing at day 30 for treatment of ulcerative colitis with a range of visilizumab doses.

FIG. 9 depicts the clinical response to treatment of Crohn's disease with visilizumab.

FIG. 10 depicts the clinical response to treatment of Crohn's disease in responding patients.

### DETAILED DESCRIPTION OF THE INVENTION

Definitions: As used herein, the term “antibody” or “immunoglobulin” is intended to encompass both polyclonal and monoclonal antibodies. The preferred antibody is a monoclonal antibody reactive with CD3. The term “antibody” is also intended to encompass mixtures of more than one antibody reactive with CD3 (e.g., a cocktail of different types of monoclonal antibodies reactive with CD3). The term “antibody” is further intended to encompass whole antibodies, biologically functional fragments thereof, single-chain antibodies, and chimeric antibodies comprising portions from more than one species, bifunctional antibodies and antibody conjugates and humanized or human antibodies. Biologically functional antibody fragments, which can also be used, are those peptide fragments derived from an antibody that are sufficient for binding to CD3.

By “a therapeutically effective” amount of a drug or pharmacologically active agent or pharmaceutical formulation is sufficient amount of the drug, agent or formulation to provide the desired effect.

A “subject,” or “patient” is used interchangeably herein, which refers to a vertebrate, preferably a mammal, more preferably a human.

The term “epitope” includes any protein determinant capable of specific binding to an immunoglobulin or an antibody. Epitopic determinants usually consist of active surface groupings of molecules such as amino acids or sugar side chains and usually have specific three-dimensional structural characteristics, as well as specific charge characteristics. Two antibodies are said to bind to the same epitope of a protein if amino acid mutations in the protein that reduce or eliminate binding of one antibody also reduce or eliminate binding of the other antibody, and/or if the antibodies compete for binding to the protein, i.e., binding of one antibody to the protein reduces or eliminates binding of the other antibody.

The term “derived from” means “obtained from” or “produced by” or “descended from”.

The term “genetically altered antibodies” means antibodies wherein the amino acid sequence has been varied from that of a native antibody. Because of the relevance of recombinant DNA techniques to this invention, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics.

The term “humanized antibody” or “humanized immunoglobulin” refers to an immunoglobulin comprising a human framework, at least one and preferably all complimentarily determining regions (CDRs) from a non-human antibody, and in which any constant region present is substantially identical to a human immunoglobulin constant region, i.e., at least about 85-90%, preferably at least 95% identical. Hence, all parts of a humanized immunoglobulin, except possibly the CDRs, are substantially identical to corresponding parts of one or more native human immunoglobulin sequences. See, e.g. Winter et al., U.S. Pat. No. 5,225,539; Queen et al., U.S. Pat. No. 6,180,370 (each of which is incorporated by reference in its entirety).

The term “chimeric antibody” refers to an antibody in which the constant region comes from an antibody of one species (typically human) and the variable region comes from an antibody of another species (typically rodent).

The present invention provides a method of treating or preventing at least one T-cell mediated disorders in a subject in need of such a treatment or prevention by specifically inhibiting the activation or proliferation, or inducing apoptosis of the activated T-cells, preferably by administering to said subject a therapeutically effective amount of an anti-CD3 antibody. In one embodiment, the T-cell mediated disorders are the conditions manifesting undesired immune responses. Such conditions include autoimmune diseases, transplant rejection, graft vs. host diseases, inflammation, allergic reactions, and sepsis. Exemplary autoimmune diseases include, but are not limited to, Addison's disease, autoimmune diseases of the ear, autoimmune diseases of the eye such as uveitis, autoimmune hepatitis, Crohn's disease, diabetes (Type I), epididymitis, glomerulonephritis, Graves' disease, Graft vs. Host disease. Guillain-Barre syndrome, Hashimoto's disease, hemolytic anemia, systemic lupus erythematosus, multiple sclerosis, myasthenia gravis, pemphigus vulgaris, psoriasis, rheumatoid arthritis, sarcoidosis, scleroderma, Sjogren's syndrome, spondyloarthropathies, thyroiditis, ulcerative colitis and vasculitis.

These T-cell mediated disorders can be treated by administering to a patient in need of such a treatment a molecule that specifically modulate activated T-cells, meaning that the molecules only have impact on activated T-cell while do not disturb the other T-cells. These T-cell modulating molecules can particularly inhibit the undesired activation and proliferation of the activated T-cells. Therefore, the treatment of the present invention is a disease modifying process. In a preferred embodiment of the present invention, the treatment will lead to a long-term remission of the disorders described herein, preferably the inflammatory bowel diseases, such as UC. In one example, these molecules are anti-CD3 antibodies.

The process of T-cell activation represents a contingent cascade of events in which each event is dependent on the expression of the previous components. During the early phase of T-cell activation, T-cells undergo enormous changes, characterized by protein phosphorylation, membrane lipid changes, ion fluxes, cyclic nucleotide alterations, increased or decreased RNA synthesis of constitutive and newly activated gene products, and cell volume increases (blast transformation). The later cellular responses, such as proliferation, generally result from a complex cascade of gene activation events and the coordinated sequential influence of the products of these genes. Ultimately, activation of the resting T-lymphocyte may be manifested in a variety ways but includes the expression of new cell surface molecules, secretion of a host of lymphokines, cell proliferation, cellular differentiation, and even programmed cell death (apoptosis). For the purpose of the present invention, activated T-cells include T-cells in any of the above-mentioned activated phases.

The various parameters are used by the one skilled in the art to assess T-cell activation. These parameters include (a) early signal transduction events, such as protein tyrosine phosphorylation or an increase in cytoplasmic free calcium ([Ca2+]), that do not necessarily lead to a cellular response; (b) expression of new cell surface activation antigens, including the a chain (CD25) of the IL-2 receptor (IL-2R), the transferrin receptor, class II MHC molecules on human T-cells, and CD69, a molecule with as yet unknown function; (c) production of lymphokines, such as IL-2 or IL-4; (d) cell proliferation; and (e) cytolytic activity. These parameters can be detected by the methods known in the art.

The present invention provides a method for the treatment of ulcerative colitis (UC) or/and other inflammatory bowel diseases such as Crohn's disease comprising administering to a patient in need thereof a therapeutically effective amount of an antibody recognizing CD3. The treatment decreases the severity of UC, prolongs the remission period of UC, reduce the frequency of relapse, or/and completely eradicate the symptoms.

The severity of UC is manifested, e.g., by the MTWSI score or MAYO score of said subject. The MTWSI is a standardized rating scale used by the treating physician to classify disease severity in UC patients (Lichtiger S., et al., N. Engl. J. Med; 330(26): 1841-1845 (1994); Truelove, S. C., et al., British Medical Journal 2: 1041(1955)). Disease symptoms are graded using individual scales for diarrhea, nocturnal diarrhea, rectal bleeding, fecal incontinence, abdominal cramping, general well being, need for antidiarrheals, and abdominal tenderness. Each category has its own scale (range 0 to 1-5) (0=normal and higher numbers reflect increasing severity); a maximum total score is 21 points. The parameters of MTWSI score are described in Table 4. Clinical response to treatment is defined as a decrease in the MTWSI score of at least 2 points to an absolute MTWSI score of less than 10 sustained for at least 30 days. Remission, including clinical remission and endoscopic remission, in these UC patients is defined as a decrease in the MTWSI score to less than or equal to 4 sustained for 60 days. A subject with an MTWSI score of greater than or equal to 11, which has failed to respond to glucocorticoid therapy has a clinically “severe” case of UC. A subject with an MTWSI score of greater than 7 and less than or equal to 10, maintained on 5-ASA±azathioprine or responsive to a short course of glucocorticoid, has a clinically “moderate” case of UC. A subject with an MTWSI score of greater than 4 and less than or equal to 7, maintained on 5-ASA, has a clinically “mild” case of UC. A subject with an MTWSI score of less than or equal to 4.

The Mayo Scoring System is another standardized rating scale used by the treating physician to classify disease severity in UC patients (Schroeder, K. W., et al., N. Engl. J. Med. 317: 1625-1629 (1987)). Disease symptoms are graded using individual scales for stool frequency, rectal bleeding, a physician's global assessment (PGA), and the findings of flexible proctosigmoidoscopy. The parameters of MAYO score are described in Table 5. A total Mayo UC activity score of 0 to 2 points indicates remission or minimally active disease; a score of 3 to 5 points indicates mildly active disease; a score of 6 to 10 points indicates moderately active disease; and a score of 11 to 12 may indicate moderate or severe disease, depending on the patient's MTWSI score.

The severity of UC is also measured by endoscopy of the colon. A severe endoscopic appearance has confluent mucosal ulcerations with purulent exudates and loss of mucosal vascular pattern and haustral architecture. A moderate endoscopic appearance has haustral edema, mucosal erythema, erosions and loss of mucosal vascular pattern. A mild endoscopic appearance has loss of mucosal vascular pattern and erythema. A normal endoscopic appearance has pink confluently visible mucosal vessels.

The methods of the present invention can be used for the treatment of mild, moderate, or/and severe ulcerative colitis, preferably, the steroid-refractory ulcerative colitis, and more preferably, the severe steroid-refractory ulcerative colitis.

When applied to a population of UC patients, treatment with anti-CD3 antibodies will lead to a reduction of at least 50% (MTWSI) or 75% (MTWSI) in the MTWSI score or even complete or near-complete clearance of the UC symptom. When applied to a population of UC patients, treatment with anti-CD3 antibodies will lead to a reduction of at least 5, 6, 7, 8, 9 or 10 in the MTWSI score. Remission, including clinical or/and endoscopic remission, should be achieved by the method of the present invention in at least 20% or 30%, but preferably 40% or 50% or even 60%, more preferably 70% or 80% and most preferably 90% or more, or even about 100% of the patients. Preferably, this effect should be demonstrated in a clinical trial, for example a phase I or phase II clinical trial, and the increase in responses or remissions relative to the control group (not treated with the anti-CD3 antibody) should be statistically significant. The MTWSI score can be measured at about 8, 15, 30, 60, 90 days, and at 6 months or 1 year after beginning or end of treatment, or at some other convenient time.

The remission can be achieved as short as no more than 20, 30, 60, 90, 120 or 150 days after the end of the treatment. Once achieved, the remission should last for at least 1, 2, 3, 4, 5, 6, 7, 8, 10 months or 1, 2, or 5 years to an indefinite period of time. Fewer numbers of the incidence of relapses or no relapses should be observed even without any other clinical treatment, such as steroid or 5-ASA treatment. In a preferred embodiment, the UC patients continue to experience clinical improvement for at least 1, 2, 4, or 6 months or 1, 2, or 5 year after the end of the treatment. Clinical improvement can be any improvement in any symptoms manifested in the parameters of the MTWSI or/and the MAYO score.

Anti-CD3 antibodies for use in the present invention include antibodies that bind to any epitope of CD3. They include natural anti-CD3 antibodies (the antibodies that are produced by a host animal) and recombinant anti-CD3 antibodies. The anti-CD3 antibodies of all species origins are included. Non-limiting exemplary natural anti-CD3 antibodies include anti-CD3 antibodies derived from human, chicken, goats, and rodents (e.g., rats, mice, hamsters and rabbits), including transgenic rodents genetically engineered to produce human antibodies (see, e.g., Lonberg et al., WO 93/12227 (1993) and Kucherlapati, et al., WO 91/10741 (1991)), which are herein incorporated by reference in their entirety). Antibodies useful in the present invention also may be made using phage display methods (see, e.g., Dower et al., WO 91/17271 and McCafferty et al., WO 92/01047, which are herein incorporated by reference in their entirety). For use in human patients, the antibodies must bind to human CD3. The antibodies should have binding affinity for CD3 of at least 10⁷ M⁻¹ but preferably at least 10⁸ M⁻¹, more preferably at least 10⁸ M⁻¹, most preferably 10⁹ M⁻¹ and ideally 10¹⁰ M⁻¹ or higher. The affinity of the antibodies may be increased by in vitro mutagenesis using phage display or other methods (see, e.g., Co, et al., U.S. Pat. No. 5,714,350, which is herein incorporated by reference in its entirety).

Preferably, the antibodies will be neutralizing, that is, they will neutralize at least one but most preferably all biological properties of CD3, for example, stimulation of T-cell proliferation. The antibodies will generally inhibit or block binding of CD3 to the T-cell receptor. The antibodies should inhibit proliferation and activation of the activated T-cells, or induce apoptosis of the activated T-cells.

Preferably, the antibodies substantially do not have the capacity to specifically bind Fcy receptors and thereby the antibodies substantially do not activate mitogenic responses in T-cells in most or all patients. Preferably, the antibodies have the following desirable properties as immunosuppressive agents: they can suppress immune responses of T-cells without inducing mitogenic activity resulting in harmful release of cytokines, at least in most (meaning at least 67%, 75%, 90% or 95% as used herein) patients. Preferably, the antibodies have one or more of the desirable properties as immunosuppressive agents as described in U.S. Pat. No. 5,834,597 (which is incorporated by reference in its entirety).

The polyclonal forms of these antibodies can be produced in non-human host animals by immunization with human CD3. The monoclonal antibodies can be produced by immunization and hybridoma methodology. The hybridoma methodology and immunization procedure are well known in the art.

Recombinant DNA techniques can be used to produce recombinant anti-CD3 antibodies, which are also included in the present invention. The amino acid sequence of such recombinant antibodies can be identical to the sequences of amino acids found in natural antibodies. Alternatively, it can be genetically altered so that the amino acid sequence has been varied from that of a native antibody. Recombinant anti-CD3 antibodies include antibodies produced by any expression systems including both prokaryotic and eukaryotic expression systems. Exemplary prokaryotic systems are bacterial systems that are typically capable of expressing exogenously introduced sequences at large quantity. Illustrative eukaryotic expression systems include fungal expression systems, viral expression systems involving eukaryotic cells such as insect cells, plant-cells and especially mammalian cells (such as CHO cells and myeloma cells such as NS0 and SP2/0) which are well-known to those of skill in the art. The antibodies may also be produced by chemical synthesis. However they are produced, the antibodies will be purified by art-known methods such as filtration, chromatography (e.g., affinity chromatography such as by protein A, cation exchange chromatography, anion exchange chromatography, and gel filtration). The minimum acceptable purity of the antibody for use in pharmaceutical formulations will be 90%, with 95% preferred, 98% more preferred and 99% or higher most preferred.

Preferably, the genetically altered anti-CD3 antibodies used in the present invention include humanized antibodies that bind to and neutralize CD3. Examples of these humanized antibodies are disclosed in U.S. Pat. Nos. 5,834,597 and 6,129,914, which are hereby incorporated by reference in its entirety. An exemplary, preferred humanized anti-CD3 antibody is visilizumab, comprising a mature light chain variable region, whose amino acid sequence is position 21 to 126 of SEQ ID NO 1, and a mature heavy chain variable region, whose amino acid sequence is position 20 to 139 of SEQ ID NO 2. Visilizumab (HuM291; Nuvion®) is a humanized IgG2 monoclonal antibody developed at Protein Design Labs, Inc. (Fremont, Calif.) (PDL) that has amino acid substitutions at position 234 and 237 in the CH2 domain of the Fc region. SEQ ID NO: 3 depicts the amino acid sequence of heavy chain constant region of visilizumab. This change is associated with a diminished release of cytokines by human peripheral blood mononuclear cells expose to the antibody in vitro, as well as reduced expression of activation markers by human peripheral blood T lymphocytes. Duo to the modified Fc region, visilizumab is capable of mediating efficient immunosuppression without causing severe cytokine release syndrome or heightened immunogenicity.

Other preferred antibodies include those that bind to the same epitope of CD3 as visilizumab (Nuvion®), especially other humanized forms of the M291 antibody described in U.S. Pat. No. 5,834,597. Preferably, the antibody that binds to the same epitope of CD3 as visilizumab has an amino acid sequence that is at least 80% identical to amino acid sequence of visilizumab. More preferably, the amino acid sequence is at least 85% identical. Even more preferably, the amino acid sequence is at least 90% identical. Even much more preferably, the amino acid sequence is at least 95% identical. Preferably, the CDR of the antibody that binds to the same epitope of CD3 as visilizumab has an amino acid sequence is at least 80% identical to the amino acid sequence of the CDR of visilizumab. More preferably, the amino acid sequence is at least 85% identical. Even more preferably, the amino acid sequence is at least 90% identical. Even much more preferably, the amino acid sequence is at least 95% identical. Most preferably, the amino acid sequence is 100% identical. The antibody may be of any of the recognized isotypes, but the four IgG isotypes are preferred, with IgG2 especially preferred. Antibodies with constant regions mutated to have reduced effector function, for example the IgG2m3 and other IgG2 mutants described in U.S. Pat. No. 5,834,597 (which is incorporated by reference in its entirety), are additional preferred choices.

The genetically altered anti-CD3 antibodies also include chimeric antibodies that bind to and neutralize CD3. Preferably, the chimeric antibodies comprise a variable region derived from a mouse or rat and a constant region derived from a human so that the chimeric antibody has a longer half-life and is less immunogenic when administered to a human subject. The method of making chimeric antibodies is known in the art.

The fragments of the above-described anti-CD3 antibodies, which retain the binding specificity to CD3, are also included in the present invention. Examples include, but are not limited to, the heavy chains, the light chains, and the variable regions as well as Fab and (Fab′)2 of the antibodies described herein.

The genetically altered antibodies also include modified anti-CD3 antibodies that are functionally equivalent to above antibodies and antibody fragments. Modified antibodies providing improved stability and/or therapeutic efficacy are preferred. Examples of modified antibodies include those with conservative substitutions of amino acid residues, and one or more deletions or additions of amino acids which do not significantly deleteriously alter the antigen binding utility. Substitutions can range from changing or modifying one or more amino acid residues to complete redesign of a region as long as the therapeutic utility is maintained. Antibodies of this invention can be can be modified post-translationally (e.g., acetylation, and phosphorylation) or can be modified synthetically (e.g., the attachment of a labeling group). Fragments of these modified antibodies that retain the binding specificity can also be used.

The present invention provides a pharmaceutical formulation comprising the antibodies described herein. Pharmaceutical formulations of antibodies are prepared for storage by mixing the antibodies having the desired degree of purity with optional physiologically acceptable carriers, excipients, or stabilizers, in the form of lyophilized or aqueous solutions. Acceptable carriers, excipients or stabilizers are nontoxic to recipients at the dosages and concentrations employed, and include buffers such as phosphate, citrate, and other organic acids; antioxidants, preservatives, low molecular weight polypeptides, proteins, hydrophilic polymers, amino acids, carbohydrates, chelating agents, sugar, and other standard ingredients known to people skilled in the art (Remington's Pharmaceutical Science 16th edition, Osol, A. Ed. 1980).

The formulation herein may also contain more than one active compound as necessary for the particular indication being treated, preferably those with complementary activities that do not adversely affect each other. Such molecules are suitably present in combination in amounts that are effective for the purpose intended.

Active ingredients of the above pharmaceutical formulation may also be entrapped in microcapsules, in colloidal drug delivery systems (for example, liposome, albumin microspheres, microemulsions, nano-particles and nanocapsules), in macroemulsions, or in sustained-release preparation. Such techniques are known to people skilled in the art (Remington's Pharmaceutical Sciences).

The formulation to be used for in vivo administration is usually stored at 2 to 80 C. The formulations often contain no preservatives and should be used within 4, 12 or 24 hours of withdrawal from the vial and dilution into saline. The formulation is preferably administered intravenously or subcutaneously with or without filtration. Preferably, humanized anti-CD3 antibody, visilizumab is stored in a single-use glass vial containing 1.0 mL of visilizumab at a concentration of 1.0 mg/mL in sterile saline buffer. However, concentrations from 1 to 10 mg/mL (e.g., 1, 2, 5 or 10), 20 to 50 mg/mL (e.g., 20, 30, 40 or 50) or 60 to 100 mg/mL (e.g., 60, 70, 80, 90 or 100) are also possible.

The antibodies prepared in a pharmaceutical formulation can be administered by any suitable route including oral, rectal, nasal, topical (including transdermal, aerosol, buccal and sublingual), parental (including subcutaneous, intramuscular, intravenous and intradermal) or by inhalation therapy. It will also be appreciated that the preferred route may vary with the condition and age of the recipient.

Preferably, the pharmaceutical formulation is delivered parentally, for example, intravenously by bolus injection, so that a therapeutically effective amount of said formulation is delivered via systemic absorption and circulation.

A therapeutically effective amount of above formulations depends on the severity of the UC, the patient's clinical history and response, and the discretion of the attending physician. The formulation is suitably administered to the patient at one time or over a series of treatments. The initial candidate dosage may be administered to a patient. The proper dosage and treatment regime can be established by monitoring the progress of therapy using conventional techniques known to the people skilled of the art.

The amount of active ingredients that may be combined with the carrier materials to produce a single dosage form will vary depending upon the subject treated and the particular mode of administration. It will be understood, however, that the specific dose level for any particular patient will depend upon a variety of factors, including the activity of the specific formulation employed, the age, body weight, general health, sex, diet, time of administration, route of administration, rate of excretion, drug combination and the severity of the particular disease undergoing therapy, and can be determined by those skilled in the art.

In particular, an exemplary effective dose for the treatment of UC between about 0.001 mg/kg to about 100 mg/kg, preferably between about 0.001 mg/kg to about 10 mg/kg, and more preferably about 0.005 mg/kg to about 0.100 mg/kg. Preferred dose levels include about 0.001 mg/kg, about 0.005 mg/kg, about 0.0075 mg/ml, about 0.010 mg/kg, about 0.015 mg/kg, and about 0.020 mg/kg. The preferred dose can be within a range of any two of the above indicated dose levels.

Depending on the progress in treatment and the physical conditions of the patients, the regimen of the treatment of UC can vary significantly. Typically, a patient is administered at least a single dose of pharmaceutical formulation comprising any one of the antibodies described herein, which is named as “the initial dose” or “the initial administering or administration” if there are any additional doses follow. The antibody drug can be administered once or multiple times at a frequency of e.g., 1, 2, 3, or 4 times per day, week, bi-weeks, every 6 weeks, or every month, or every 2, 3, or 6 months. The duration of the treatment of one treatment course should last for at least one or two days, such as, one to several (2, 3, 4, 5, or 6) days, weeks, months or years, or indefinite, depending upon the nature and severity of the disease. The duration of the treatment is calculated as the period from the initial administration of the antibodies to the last administration of the antibodies. The patient may receive 2, 3, 4 or more courses of treatment if the disease relapses. The frequency of the administration can be adjusted according to the improvement progress of the patients.

A preferred dose for both UC and Crohn's disease is 1-15, 1-10, 1-5, 5-10, 5-15, 10-15, 4-6, 5, 8-12, 10, 2.5, 2.5-5, 2.5-7.5 or 2.5-10 μg antibody/kg patient body weight. The dose is usually administered once or twice per relapse-remission cycle of disease (e.g., one or two doses per relapse). If administered twice, the dose is preferably administered on consecutive dose. For selecting dose on a patient level, the cumulative dose given on two consecutive days in a two-dose regime should be equivalent to the single dose given in a one-dose regime. Administration of a divided dose on consecutive days may be advantageous in reducing cytokine release symptoms but is otherwise less convenient for the patient. Spacing of two doses by a single day is optimal in view of the short half-life of visilizumab when administered at the low dosages mentioned above (about 17 hours). However, increasing the interval much beyond a day loses the effect of a bolus injection because of the short half-life of visilizumab at the low dosages being employed. As shown in the Examples, the methods are effective notwithstanding disease that is refractory to treatment with intravenous corticosteroids, anti-TNFalpha antibodies or in patients having an EBV titer of greater than 5000 copies of DNA per ml plasma.

As an alternative to calculating dosage on a mg/kg basis, antibody can be formulated for single-dose administration using 75 kg as a typical adult weight. Doses calculated by multiplying the above mg/kg figures by 75 kg to convert to mass doses can be rounded to the nearest 25, 50 or 100 μg. Single-dose formulations can thus contain, for example, 50, 75, 100, 200, 250, 500, 750, 1000 or 1250 μg antibody.

As a preferred treatment regimen for UC, a dose of anti-CD3 antibody is administered to a patient as one daily bolus injection on each of the two consecutive days.

To reduce the infusion-related symptoms, the pharmaceutical formulation comprising anti-CD3 antibodies can also be used as separately administered formulations given in conjunction with other agents. Typically, these agents include methyprednisolone, hydrocortisone, ondansetron, acetaminophen, and numerous additional agents that have the similar functions and are well-known to those skilled in the art. These other agents can be administered by any suitable route including oral, rectal, nasal, topical, parental (including subcutaneous, intramuscular, intravenous and intradermal), or by inhalation therapy.

The dose levels of these agents are also known in the art, for example, from 1 mg to 100 g per patient. Exemplary doses include 10-50 mg, 60-200 mg, or 200-500 mg for methyprednisolone, hydrocortisone and ondansetron; and 100-500 mg, 600-1000 mg, 1-5 g for acetaminophen. Single or multiple additional immunomodulating agents can be administered to the patients, for example, at least about 1, 2, 3, 4, 5, 6, 7, 8, 10, 12, 14, 20, 24, 36 hours or 2, 3, 4, 5, 7, 10, 20, 40, or 60 days, prior to or/and after the initial or/and each administering of the pharmaceutical formulation of anti-CD3 antibodies.

In one example, the patients are pre-treated with methyprednisolone (or hydrocortisone) and ondansetron about 1 hour prior to receiving the first dose of the antibodies, for example, about 50 mg methyprednisolone and ondansetron intravenously. In another example, the patients receive acetaminiophen about 1 to 2 hours after receiving each administering of anti-CD3 antibodies, for instance, about 1000 mg acetaminiophen orally. In a preferred example, the patients are both pre-treated with methyprednisolone and ondansetron and receive acetaminophen after receiving each administering of anti-CD3 antibodies as described in these two exemplary embodiments.

The methods of the present invention lead to superior clinical efficacy (about 100% remissions in the treated patients) for treating UC or other inflammatory bowel diseases, especially for the severe steroid-resistant ulcerative colitis. The methods can be used alone or in combination with any other treatment courses. For example, patients who are undergoing the conventional treatment can be subject to the antibody treatment regimens described herein simultaneously until the desired efficacy is accomplished. In one example, the patients who are undergoing a treatment of steroids or other agents as listed in Table 3 are subject to the antibody treatment regimens described herein. The patients should receive the steroids for at least 1, 2, 3, 5, 7, 10, 20, 30, or 90 days before the onset of the anti-CD3 antibody regimens (the initial administering of the antibody pharmaceutical formulation). The patients will continue to receive the steroid at least about 1 day (for example, about 5, 7, 10, 15, 20, or 50 days), after receiving the last administration of the anti-CD3 antibodies. Patients will continue receiving any other immunomodulating agents that are part of their current treatment regimen. The dosage range will be decided by the treating physician, for example, usually from 1 mg/kg to 100 mg/kg for steroid or 5-ABA.

For efficacy of the treatment of UC, patients are scored for MTWSI and MAYO. Colon biopsy materials are evaluated for inflammatory activities. Any surgical interventions will be documented.

The following examples are offered by way of illustration and not by way of limitation. The disclosure of all citations in the specification is expressly incorporated herein by reference for all purposes.

### EXAMPLE 1

This example describes the study synopsis of the Phase I, dose-escalation, pilot study of visilizumab in patients with severe ulcerative colitis that is refractory corticosteroids.

### Study Synopsis

### A Phase I, Dose-Escalation, Pilot Study of Visilizumab in Patients With Severe Ulcerative Colitis that is Refractory to Corticosteroids

Protocol Number: 291-406, Amendment B
Phase: I
Study Drug: Visilizumab (Nuvion®; HuM291)
Indication: Ulcerative colitis
Regulatory Status US IND No. 9443
Study Design
   Dose-escalation, pilot study designed to obtain safety, tolerability, and preliminary efficacy data. Two stages were planned for this study. In Stage 1, consecutive groups of up to 10 patients was treated with 2 IV doses of visilizumab at 4 escalating dose levels until the maximum tolerated dose (MTD) or Optimum Biological Dose (OBD) is reached. Dose escalation would not occur until Day 30 safety and efficacy data were obtained on all patients in the current dose level. If necessary, de-escalation to 2 dose levels below Dose Level I would also be considered during Stage 1. In Stage 2, up to 20 additional patients would be enrolled at the OBD or MTD.
Patient Population
   Men and women, 18 to 70 years of age, with severe ulcerative colitis (UC) that has failed to respond to intravenous (IV) steroid therapy
Inclusion Criteria
   A diagnosis of UC verified by colonoscopy or barium enema performed within 36 months prior to study entry.
   Active disease documented by a MTWSI score of 11 to 21, despite an ongoing treatment course of IV steroids for a minimum of 5 days prior to study entry.
Route of Administration:
   Intravenous (IV) by bolus injection
Dose Levels:
   Dose Level 1*: 15 μg/kg q.d. administered on Days 1 and 2
   Dose Level 2: 30 μg/kg q.d. administered on Days 1 and 2
   Dose Level 3: 45 μg/kg q.d. administered on Days 1 and 2
   Dose Level 4: 60 μg/kg q.d. administered on Days 1 and 2
      * In the event that it was necessary to deescalate from Dose Level 1, the following two dose levels may be used:
   10 μg/kg q.d. administered on Days 1 and 2
   7.5 μg/kg q.d. administered on Days 1 and 2
   See Section 3.5 for dose-escalation and de-escalation guidelines, and for definition of OBD and MTD.
Dosage Form and Strength:
   1.0 mg/mL in sterile saline solution
Visilizumab Storage:
   Visilizumab should be stored under controlled, refrigerated conditions at 2 to 8° C. (36 to 46° F.). The formulation contains no preservatives and should be used within 12 hours of withdrawal from the vial.
Pre- and Postmedications
   1 hour before receiving the first dose of visilizumab: 50 mg of methylprednisolone IV (or equivalent dose of hydrocortisone IV), and ondansetron (Zofran™).
   1 to 2 hours after each treatment with visilizumab: 1000 mg of acetaminophen.
   Patients continued to receive corticosteroids according to their current regimen for a period of at least 7 days after receiving visilizumab. After 7 days, corticosteroid regimens may be continued or tapered. Patients continued to receive any other immunomodulatory agents that were part of their current treatment regimen.
Duration of Treatment and Follow Up
   Screening (baseline tests) took place up to 3 weeks prior to visilizumab dosing. Dosing occurred on Days 1 and 2, and follow-up visits were scheduled for Days 8, 15, 30, 60, and 90, and at 6 months. At 6 months and 1 year, patients should follow for long-term safety information; at 6 months they should also be followed up for efficacy information (see below).
Number of Sites/Sample Size
   This study would take place at up to 10 centers in the US. It is anticipated that up to 60 UC patients (up to 10 at each of the four levels, and up to 20 more at the OBD or MTD) would be enrolled in this trial.
Statistical Methods
   Descriptive statistics and 95% confidence intervals would be employed where appropriate. Tabulations and listings would summarize the characteristics of the patient population. Pharmacokinetic (PK) and pharmacodynamic (PD) results were presented by dose level in tables and graphs, without formal statistical testing of between-group differences. Antibody responses were noted and AEs will be tabulated. MTWSI and Mayo System scores, and their corresponding changes over time, were summarized and listed.
Primary Objectives
   To evaluate the safety and tolerability of visilizumab when administered to patients with severe UC that is refractory to steroids.
Secondary Objectives
   1) To determine the maximum tolerated dose (MTD) or optimum biological dose (OBD) of visilizumab in this study.
   2) To obtain preliminary evidence of biological activity in this indication.
   3) To determine relationships between pharmacokinetics and pharmacodynamics of visilizumab, clinical response, and toxicity.
Safety Measurements
   Adverse Events (AEs) and Serious Adverse Events (SAEs) were documented through Day 60, and were characterized according to severity and relationship to study drug. Concomitant medications were documented through Day 60. Patients were followed up for opportunistic infections and malignancies at 6 months and 1 year after treatment.
   Laboratory values of all patients were monitored (serum chemistry up to Day 15, and hematology up to Day 30). If additional blood samples were required past Day 30 to document T-cell recovery, additional blood samples would be drawn at the same times for hematology and PK samples.
   Epstein Barr virus (EBV) DNA copy number were monitored in all patients using blood samples drawn at baseline and on Days 8, 15 and 30. If the EBV titer on Day 30 is above the patient's baseline level, EBV assays were repeated every 2 weeks until it returns to baseline.
Efficacy Measurements
   Disease activity (severity of symptoms) was measured at baseline, at 1 day, at 2 weeks, and at 1, 2, 3, and 6 months after visilizumab dosing using the MTWSI scoring system. In addition, patients' UC symptoms was assessed at baseline and at the 1-month follow-up visit using the Mayo Scoring system. Patients also underwent flexible sigmoidoscopies at baseline and at one month; colon biopsy samples were examined for pathology. Any surgical interventions were documented.
Pharmacodynamic Measurements
   Circulating CD3⁺CD4⁺T-cell counts were monitored in all patients at a minimum through Day 30. After that, T-cell data would be collected from patients every 7 days until recovery was documented. Adequate T-cell recovery is defined as ≧200 cells/μL or >50% of patient's baseline value.
Pharmacokinetic Measurements
   A pharmacokinetic (PK) profile was determined for each patient, using blood samples drawn before and after visilizumab dosing on Days 1 and 2, and again on Days 8, 15, 30, and 90. If additional blood samples were required past Day 30 to document T-cell recovery, additional serum samples would be drawn at the same times for PK assays.
Anti-Ab Assessments
   Patients were evaluated during the study for development of antibodies to visilizumab (Anti-Abs) using blood samples drawn prior to dosing on Day 1, and again on Days 15, 30, and 90.

### EXAMPLE 2

This example describes the detailed protocols of the Phase 1, dose-escalation, pilot study of visilizumab in patients with severe ulcerative colitis that is refractory corticosteroids.

### 1. OBJECTIVES OF STUDY

### 1.1. Primary Objective

To evaluate the safety and tolerability of visilizumab when administered to patients with severe ulcerative colitis that is refractory to steroids.

### 1.2. Secondary Objectives

1) To determine the maximum tolerated dose (MTD) or optimum biological dose (OBD) of visilizumab in this study.
2) To obtain preliminary evidence of biological activity in this indication. This may be signaled by a decrease in the MTWSI score, or the Mayo Score (both of which reflect disease symptom severity), and by lowered incidence of surgical intervention.
3) To determine relationships between pharmacokinetics and pharmacodynamics of visilizumab, clinical response, and toxicity.

### 2. STUDY DESIGN AND METHODS

### 2.1. Design and Controls

This is a Phase I, dose-escalation, pilot study to be conducted at up to 10 centers in the U.S. Up to 60 patients with severe UC would be enrolled in this trial. Two stages were planned for this study. In Stage 1, consecutive groups of up to 10 patients each would be treated with 2 IV doses of visilizumab at one of 4 escalating dose levels until the MTD or OBD is reached. Dose escalation would not occur until Day 30 safety and efficacy data are obtained on all patients in the current dose level. If necessary, de-escalation to 2 dose levels below Dose Level I would also be considered during Stage 1. In Stage 2, up to 20 additional patients would be enrolled at the OBD or MTD.

### 2.2. Patient Assignment Methods

Patients who meet the eligibility criteria at screening and provide a written informed consent were enrolled in the study. The principal investigator or designee would fax the completed enrollment authorization case report forms (CRF) to PDL (see page ii, Patient Enrollment Assignments, for names of individuals to contact). Upon receipt of these CRFs, eligible patients were assigned an identification (I.D.) number. The assigned patient numbers reflected the corresponding site and the order in which the patients were enrolled.

### 2.3. Treatment Regimen

Patients received visilizumab at one of four dose levels (Dose Levels 1 to 4) administered as one daily IV bolus injection on each of two consecutive days (q24H) (Table 1).

TABLE 1
Dose Level Assignments

| | | Number of |
|---|---|---|
| Dose Level | Visilizumab Doses^{a} | Patients^{b} |
| 1 | 15 μg/kg | 10 |
| 2 | 30 μg/kg | Up to 10 |
| 3 | 45 μg/kg | Up to 10 |
| 4 | 60 μg/kg | Up to 10 |

| | |
|---|---|
| Maximum Total Number of Patients: | Up to 60^{b} |

^{a}If it is necessary to deescalate from Dose Level 1, a provision is made for two lower dose levels: 10 μg/kg and 7.5 μg/kg; up to 10 patients could be enrolled at each de-escalation dose level.
^{b}Up to 20 additional patients will be enrolled at the OBD or MTD.

### 2.4. Pre- and Postmedications

One hour prior to receiving the first dose of visilizumab (on Day 1), all patients were be pretreated with 50 mg of methylprednisolone IV (or an equivalent dose of hydrocortisone IV), and ondansetron (Zofran™; 32 mg IV or up to 16 mg PO), as tolerated.

All patients received 1000 mg of acetaminophen PO, 1 to 2 hours after receiving each dose of visilizumab (Days 1 and 2). In the event that constitutional symptoms occur subsequent to the administration of visilizumab, appropriate therapies may be prescribed at the discretion of the investigator. Patients continued to receive corticosteroids (same dose as prior to beginning study) for a period of at least 7 days after receiving visilizumab. After 7 days, corticosteroid regimens may then be continued or tapered, as medically indicated. Patients continued receiving any other immunomodulating agents that are part of their current treatment regimen.

### 2.5. Dose-escalation, De-escalation, and Stopping Rules

Two stages were planned for this study. In Stage 1, consecutive groups of up to 10 patients each were treated with 2 IV doses of visilizumab at one of 4 escalating dose levels until the MTD or OBD was reached. The MTD was the next dose level lower than the dose level where 2 or more patients experience a DLT or an inadequate CD3⁺CD4⁺ T-cell recovery (defined below). The OBD is defined as the lowest dose at which the most patients experience remission (for ≧1 month) and the fewest number of patients experience a DLT or an inadequate CD3⁺CD4⁺ T-cell recovery. Once the OBD or MTD was determined, up to 20 more patients would be added at that dose level (Stage 2).

A DLT is defined as an acute toxicity of Grade 3 or higher severity, related to administration of study drug. Adequate CD3⁺CD4⁺ T-cell recovery is defined as ≧200 CD3⁺CD4⁺ cells/μL, or >50% of patient's baseline value, by 4 weeks after receiving study drug.

Dose escalation would not occur until 1-month safety and efficacy data were obtained on all patients in the current dose level. De-escalation would occur immediately if 2 or more patients in the current dose group experienced a DLT and/or delayed CD3⁺CD4⁺ T-cell recovery. A provision was made for de-escalation to two dose levels below Dose Level 1 if appropriate. The conditions for dose escalation, de-escalation, and entry into Stage 2 of enrollment, and stopping rules were outlined in Table 2 below.
Notes:
   If data obtained from the first 10 patients enrolled in Dose Level 1 (15 μg/kg) indicated that this might be the OBD, a provision would be made to delay the declaration of Dose Level 1 as the OBD until 1-month safety and efficacy data were also obtained on up to 10 patients at the next lower dose level (10 μg/kg). (See Table 2.) At that point, the sponsor and investigators would review and discuss the data from both dose levels and then decide which level would be the OBD.

During Stage 2, additional DLTs would be reviewed by the sponsor as they occur, and appropriate actions would be taken in the event of unacceptable toxicity. If there were no new toxicity concerns observed during Stage 2, but it became apparent that <80% of the patients who had a clinical response at 1 month continue to have a favorable response at 3 months, the sponsor would consider dose escalation.

TABLE 2
Dose-Escalation, De-escalation, and Stopping Rules

| No. of Patients at Current | No. of Patients at | |
|---|---|---|
| Dose Level with a DLT | Current Dose Level | |
| and/or Delayed CD3⁺CD4⁺ | with a Durable Clinical | |
| T-Cell Recovery | Response at 30 Days | Instruction |
| 0 or 1 | 8 or fewer | Continue to enroll patients at the |
| | | current dose level, up to the full |
| | | cohort of 10. |
| | | Then, enroll up to 10 patients at |
| | | the next higher dose level.^{a} |
| 0 or 1 | 9 or 10 | Continue to enroll patients at the |
| | | current dose level, up to the full |
| | | cohort of 10. |
| | | Then: |
| | | If the current dose level is ≧30 μg/kg, |
| | | this is the OBD. |
| | | If the current dose level is 15 μg/kg |
| | | (Dose Level 1), enroll up |
| | | to 10 patients at the first de- |
| | | escalation dose (10 μg/kg).^{b} |
| | | Once the OBD was established, |
| | | enter up to 20 additional patients |
| | | at the OBD. |
| 2 or more | N/A | Immediately stop enrolling |
| | | patients at the current dose level. |
| | | Begin enrolling new cohort of up |
| | | to 10 patients at the next lower |
| | | dose level.^{c} |

^{a}If the current dose level is the highest planned dose level (ie, 60 μg/kg) or the OBD or MTD has been reached, enroll up to 20 additional patients at the current dose level.
^{b}Once safety and efficacy data have been obtained on patients at the 10 μg/kg dose level, the sponsor and investigators will discuss the results and decide whether the OBD will be 10 or 15 μg/kg.
^{c}If the current dose level is the lowest planned dose level (ie, 7.5 μg/kg), no additional patients will be enrolled into the study.
N/A = Not applicable

### 3. PATIENT SELECTION

### 3.1. Study Population

Up to 60 patients with severe, steroid-refractory UC were enrolled in this study. All patients must currently be on a course of steroid treatment (as described in Section 4.2 below) and be able to continue this therapy for at least 1 week after receiving visilizumab.

### 3.2. Inclusion Criteria

Patients were considered for inclusion in this study if they met all of the following criteria:
1) Male or female, 18 to 70 years of age.
2) A diagnosis of UC verified by colonoscopy or barium enema performed within 36 months prior to study entry.
3) Active disease documented by a MTWSI score of 11 to 21, despite an ongoing treatment course of IV steroids for a minimum of 5 days prior to study entry.
4) If patient is a male or female of reproductive potential, he or she agrees to use adequate contraception during the study and for 3 months after receiving visilizumab.
5) Women of childbearing potential who have a negative pregnancy test (urine or serum) at baseline screening.
6) Patients must have tested negative for Clostridium difficile within 30 days prior to study entry.
7) Patients who are capable of understanding the purpose and risks of the study and who sign an informed consent for the study.

### 4. PROCEDURES

Once preliminary eligibility was established by history, chart inspection, and routine evaluations, and a signed informed consent was obtained from the patient, the investigator or designee would contact PDL for a patient identification code number and dose level assignment.

Patients were expected to participate for up to 1 year. Screening (baseline testing) took place up to 3 weeks prior to visilizumab dosing. Dosing occurred on Days 1 and 2, and follow-up visits were scheduled for Days 8, 15, 30, 60, and 90, and at 6 months. Patients would undergo, flexible sigmoidoscopies at baseline and at the Day 30 visit. Long-term safety information was collected at 6 months and 1 year; patients had the option of being contacted by telephone for the 1-year follow up.

### 4.1. Baseline

Baseline tests must be performed within 3 weeks prior to the administration of visilizumab, unless otherwise specified below. The investigator must know the baseline test results before the first dose of visilizumab was administered, unless permission was obtained from the medical monitor at PDL. Specific evaluations used to determine patient eligibility were outlined below.
1) Medical history, including demographic information.
2) Physical examination to include height, weight, and vital signs (blood pressure, pulse rate, respiratory rate, and body temperature).
3) Neurological Exam.
4) Recording of concomitant medications taken within 3 weeks prior to dosing.
5) Flexible sigmoidoscopy with biopsy. Biopsy will be sent to pathology to rule out cytomegalovirus (CMV) inclusion bodies. Photographs will be taken of the lesion.
6) Assessment of severity of patient's UC symptoms using the MTWSI (see Table 4, Modified Truelove and Witts Severity Index) and Mayo (see Table 5) scoring systems.
7) Chest x-ray, EKG.
8) CBC with differential and platelet count.
9) Serum chemistry panel, including BUN, creatinine, total protein, albumin, total bilirubin, direct bilirubin (if total abnormally elevated), alkaline phosphatase, GGT, ALT (SGPT), AST (SGOT), glucose, calcium, phosphorous, sodium, magnesium, potassium, chloride, and carbon dioxide.
10) Blood draw for flow cytometry (T-cell) analysis.
11) Serology for human immunodeficiency virus (HIV-1 ) antibody, hepatitis B virus (HBV) surface antigen, hepatitis C virus (HCV) antibody, and CMV IgM, if not performed within 6 months prior to study enrollment.
12) Blood draw for EBV test.
13) Urinalysis (dipstick, microscopic if abnormal).
14) Urine or serum pregnancy test for females of reproductive potential.

### 4.2. Treatment: Study Days 1 and 2

The following tests and evaluations were performed on Days 1 and 2, unless specified otherwise below:
1) Blood draws for hematology (CBC with differential and platelet counts) was taken on Days 1 and 2 at the same times that blood was drawn for flow cytometry samples. Hematology blood draws occurred on Day 1: 15 min prior to visilizumab dosing and 1.0 hr after dosing; and on Day 2: 15 min prior to dosing.
2) Serum chemistry panel within 24 hours prior to the administration of visilizumab on Day 1 only.
3) Westergren erythrocyte sedimentation rate (ESR) within 24 hours prior to the administration of visilizumab on Day 1 only.
4) MTWSI evaluation prior to dosing on Day 1 only (see Table 4).
5) Vital signs (blood pressure, pulse rate, respiratory rate, and body temperature) taken at the following times on Days 1 and 2: 15 min prior to visilizumab injection; and 30 min, 1 hour, 2 hours, and 6 hours after injection. (On Day 2, the last monitoring may take place 4 to 6 hours after visilizumab injection.)
6) Recording of concomitant medications on Days 1 and 2.
7) Recording of AEs and SAEs on Days 1 and 2.
8) Blood draws for PK determinations on Day 1: 15 minutes prior to visilizumab dosing, and 1.0 and 4.0 hours after dosing; Day 2: 15 minutes prior to visilizumab dosing, and 1.0 and 6.0 hours after dosing.
9) Blood draw for immunogenicity (Anti-Ab) assay: 15 minutes prior to visilizumab dosing on Day 1 only.
10) Blood draws for flow cytometry (T-cell) analyses on Day 1: 15 minutes prior to visilizumab dosing, and 1.0 hour after dosing; Day 2: 15 minutes prior to visilizumab dosing.

### 4.3. Follow Up: Days 8, 15, and 30

The following tests and evaluations were performed on Day 8±1 day, Day 15±1 day, and Day 30±2 days, unless specified otherwise below. Note: AEs, SAEs, and concomitant medications were reported by the patient and collected at any time throughout the 30-day follow-up period, not just on the indicated visit days.
1) Blood draws for hematology (CBC with differential and platelet counts) on Days 8, 15, and 30, at the same times that blood was drawn for flow cytometry samples. If the patient's CD3⁺CD4⁺ T-cell count did not return to ≧200 cells/μL or >50% of patient's baseline value by Day 30, hematology blood draws would be repeated every 7 days (ie, Days 37, 44, etc.), in accordance with the continued blood draws for flow cytometry, until this T-cell level had been reached.
2) Serum chemistry panel on Day 15 only.
3) Blood draws for EBV testing on Days 8, 15, and 30. If the EBV titer on Day 30 was above the patient's baseline level, EBV assays would be repeated every 2 weeks until it returns to baseline.
4) Erythrocyte sedimentation rate (ESR) on Days 8, 15, and 30.
5) Recording of concomitant medications on Days 8, 15, and 30.
6) Recording of AEs and SAEs on Days 8, 15, and 30.
7) Flexible sigmoidoscopy, with photographs of any residual lesions, on Day 30 only.
8) MTWSI on study Days 15 and 30 only. Mayo Score on Day 30 only.
9) Blood draws for flow cytometry (T-cell) analyses on Days 8, 15, and 30. If the patient's CD3⁺CD4⁺ T-cell count has not returned to ≧200 cells/μL or >50% of patient's baseline value by Day 30, continue flow cytometry sampling every 7 days (ie, Days 37, 44, etc.) until this T-cell level has been reached.
10) Blood draws for PK determinations on Days 8, 15, and 30. If the patient's CD3⁺CD4⁺ T-cell count did not return to ≧200 cells/μL or >50% of patient's baseline value by Day 30, continue PK sampling every 7 days (ie, Days 37, 44, etc.), in accordance with the continued blood draws for flow cytometry, until this T-cell level was reached.
11) Blood draw for immunogenicity (Anti-Ab) analysis on Days 15 and 30 only.

### 4.4. Follow Up: Days 60 and 90

The following tests and evaluations were performed on Day 60±4 days and Day 90±4 days unless specified otherwise below. Note: AEs, SAEs, and concomitant medications may be reported by the patient and collected at any time throughout the 60-day follow-up period, not just at the scheduled Day 60 visit.
1) Recording of concomitant medications on Day 60 only.
2) Recording of AEs and SAEs on Day 60 only.
3) MTWSI on Days 60 and 90.
4) Blood draw for PK determination and immunogenicity (Anti-Ab) analysis on Day 90 only.

### 4.5. Long-Term Follow Up: 6 Months and 1 Year

At the 6-month follow-up visit, patients' UC symptoms were evaluated using the MTWSI questionnaire (see Table 4).

Patients were questioned at the 6-month and 1-year follow up to determine whether they had developed any opportunistic infections or malignancies, and whether their disease required surgical intervention. At the 1-year follow-up, patients may be contacted via a study site visit or by telephone.

### 5. MATERIALS AND SUPPLIES

### 5.1. Supplies

PDL supplied the study drug in single-use vials containing visilizumab (1.0 mg/mL) in a solution consisting of 20 mM sodium citrate, 120 mM sodium chloride, and 0.01% polysorbate 80, at pH 6.0. The vials contain approximately 1.0 mL of solution.

### 5.2. Route of Administration

Visilizumab was administered IV as a bolus injection. Care should be taken to prevent extravasation of the solution; a local inflammatory response of erythema, swelling, induration, stiffness, and pain was reported following infiltration of visilizumab upon IV injection.

One hour prior to receiving the first dose of visilizumab (on Day 1), all patients were pretreated with 50 mg of methylprednisolone IV (or an equivalent dose of hydrocortisone IV), and ondansetron (Zofran™; 32 mg IV or up to 16 mg PO), as tolerated.

Visilizumab was administered by bolus IV injection (not to exceed 1 minute). Visilizumab was not administered in conjunction with other drug solutions.

All patients received 1000 mg of acetaminophen PO, 1 to 2 hours after receiving each dose of visilizumab (Days 1 and 2). Patients continued to receive corticosteroids for at least 7 days after receiving visilizumab. After 7 days, corticoid regimens may be continued or tapered. Patients continued to receive any other immunosuppressive agents that are part of their current treatment regimen. Visilizumab was administered in the following manner:
Attach a butterfly infusion set to the syringe.
Insert the butterfly needle into the patient's vein or into a venous cannula that is patent.
Deliver visilizumab as a bolus injection not to exceed one minute.
Remove visilizumab syringe from the butterfly line and replace it with a syringe containing 5 mL of normal saline.
Deliver saline to flush the butterfly line.
Dispose syringes (and infusion set) per hospital protocol.

### 5.3. Storage of Visilizumab

Visilizumab was to be stored under controlled, refrigerated conditions at 2 to 8° C. (36 to 46° F.). Since the formulation contains no preservative, visilizumab should be administered within 12 hours of withdrawal from the vial.

Records showing the temperature of the drug storage unit were maintained at the clinical site.

### 6. Management of Intercurrent Events

### 6.1. Apparent Toxicity

Comprehensive assessments of any apparent toxicity experienced by the patient will be performed throughout the course of the study. Study site personnel will report any clinical AE, whether it is observed by the investigator or the patient (see Section 6.2, Adverse Events, for further details regarding the definition, management, and reporting of AEs).

6.1.1. Grading of Toxicity

Clinical AEs or laboratory test results will be assessed in accordance with the grading scale established by the National Cancer Institute Common Toxicity Criteria (NCI CTC) version 2 (http://ctep.info.nih.gov/CTC3.ctc.htm). Pre-existing colitis symptoms (e.g., hematochezia, diarrhea, and other symptoms of GI distress associated with the underlying disease process of inflammatory bowel diseases, such as UC) will be recorded only if they worsen from the patient's baseline assessment. These symptoms will not trigger SAE reporting, or affect dose escalation/de-escalation, unless they worsen by two or more severity grade levels, compared with the patient's baseline evaluation, and satisfy the criteria for defining an SAE (Section 6.2.1.1). SAE reporting may still occur for AEs that worsen by only one severity grade, if the event is considered by the investigator or sponsor to be related to the study drug (Section 7.2.3). See Appendix D, for a severity grading scale that can be used for clinical symptoms not listed in the NCI CTC tables. Only clinically significant abnormal lab results will be recorded as AEs.

6.1.2. Monitoring and Treatment of Toxicity

The investigator, subinvestigator, or designated health professional must be present during visilizumab administration and for the evaluation and treatment of any AE. This will be documented in the study record.

### 6.2. Adverse Events

The investigator will assess the seriousness, intensity, and causality of an AE based on the following definitions:

6.2.1. Defining Adverse Events

An adverse event (AE) is any undesirable event occurring to or in a patient enrolled in a clinical trial, whether or not the event is considered related to the test drug. This includes the time periods during which no medication is administered to a patient, such as run-in, washout, or follow-up periods. AEs include the following types of changes:
Suspected adverse drug reactions.
Other medical experiences, regardless of their relationship to the test drug, such as injury, surgery, accidents, extensions of symptoms or apparently unrelated illnesses, and significant abnormalities in clinical laboratory values, physiological testing, or physical examination findings.

6.2.1.1. Serious Adverse Events

A serious adverse event (SAE) is any adverse drug experience that occurs at any dose and results in any of the following outcomes:
Death. This includes any death that occurs during the conduct of a clinical study, including deaths that appear to be completely unrelated to the test drug (e.g., a car accident). If a patient dies during the study, and an autopsy is performed, the autopsy results will be attached to the patient's Case Report Form (CRF). Possible evidence of organ toxicity and the potential relationship of the toxicity to the test drug are of particular interest. The autopsy report should distinguish the relationship between the underlying diseases, their side effects, and the cause of death.
Life-threatening adverse drug experience. This includes any AE during which the patient is, in the view of the investigator, at immediate risk of death from the reaction as it occurs. This definition does not include any event that may have caused death if it had occurred in a more serious form.
Persistent or significant disability or incapacity.
Inpatient hospitalization or prolongation of existing hospitalization.
Congenital anomaly or birth defect.
Other medically important event that, according to appropriate medical judgment, may require medical or surgical intervention to prevent one of the outcomes listed above.

6.2.1.2. Nonserious Adverse Events

A nonserious AE includes any AE that is not described in the previous SAE category.

6.2.1.3. Unexpected Adverse Events

An unexpected AE is any AE that is not identified in nature, severity, or frequency, in the Investigator's Brochure for the current study.

6.2.2. Documenting All Adverse Events

All AEs that occur on Day 1 (following dosing with visilizumab) through Day 60 (±4 days) must be recorded accurately on the Adverse Event page of the patient's CRF. Record the date of onset and duration of the AE, and grade the severity of each sign or symptom on a scale of 1 to 5 (1=mild; 2=moderate; 3=severe; 4=life-threatening; and 5=death related to the AE), according to the NCI CTC (see Section 6.1.1). The severity of AEs that are not listed under the NCI CTC will also be classified according to the same scale. Record the treatment used and the outcome of the event. If the AE continues, mark the Adverse Event page accordingly. The investigator must attempt to explain the relationship of each AE to the test drug (unrelated, possibly related, probably related, or related).

6.2.3. Reporting and Documenting Serious Adverse Events

SAEs that occur within the period of time from administration of visilizumab on Day 1 through Day 60 (±4 days) must be reported. (See Section 6.1.1 for exceptions.) The following steps will be taken to report promptly and document accurately any SAE, even if it may not appear to be related to the test drug:
1) Report the SAE to PDL by telephone or telefax within 24 hours of a patient notifying study personnel of experiencing an SAE.
2) Record the SAE accurately on the Adverse Event page of the patient's CRF, as described in Section 6.2.2 above.
3) Submit all known patient information to PDL by fax or telephone within 24 hours of SAE occurrence on the patient's Serious Adverse Event Report. Date and sign each report before submission. Provide updated reports as new information becomes known. The following complete information must be collected:
   Study protocol number and indication
   Study site and investigator's identification
   Name of study drug and whether or not the study is blinded
   Patient's study ID (identification code and initials), age or date of birth, and sex
   Patient's weight or body surface area
   Date of randomization (if applicable)
   Description of SAE, including date of onset and duration, severity, and outcome
   Dose and total number of doses of study drug administered to patient
   Date of first and most recent (last) dose administered
   Route of administration of study drug
   Length of time from study drug administration to SAE onset
   Action taken regarding study drug administration
   Relationship of SAE to study drug
   Concomitant medications, including regimen and indication
   Intervention, including concomitant medications, used to treat SAE
   Pertinent laboratory data/diagnostic tests conducted and date
   Pertinent medical history of patient
   Date of hospital admission/discharge
   Date of death (if applicable)
4) Perform appropriate diagnostic tests and therapeutic measures, and submit all follow-up substantiating data, such as diagnostic test reports, to PDL.
5) Conduct appropriate consultation and follow-up evaluations until the events are resolved or otherwise explained by the principal investigator.
6) Review each SAE report with PDL and evaluate the relationship of the SAE to study drug treatment and to the underlying disease. PDL will determine whether the SAE is unexpected in nature.
7) Based on a cooperative assessment of the AE with PDL, a decision for any further action will be made. The primary consideration is patient safety. If the discovery of a new AE related to the study drug raises concern over the safety of its continued administration to patients, PDL will take immediate steps to notify the FDA and all participating investigators in this study.
8) The investigator must report all SAEs and unexpected problems promptly to his/her Institutional Review Board (IRB) if these events represent a significant risk to the patients. (See FDA ICH Guidelines, GCP E6, Section 4.11.1; this information can be accessed at: www.fda.gov/cder/guidance/959fnl.pdf.)
9) PDL may determine that other actions are required, including one or more of the following:
   1) Alteration of existing research by modification of the protocol.
   2) Discontinuation or suspension of the study.
   3) Alteration of the informed consent process by modification of the existing consent form informing current study participants of new findings.
   4) Modification of the Investigator's Brochure to include AEs newly identified as expected and/or study drug-related, if appropriate.
      6.2.4. Follow Up of Adverse Events

All AEs are followed until they are resolved or otherwise explained by the principal investigator.

### 6.3. Concomitant Medications

The concomitant medications listed in Table 3 are allowed, for treatment of ulcerative colitis. Patients should continue their regimen of corticosteroids for 1 week following dosing of visilizumab. After 1 week, these can be tapered at the discretion of the treating physician.

Record all concomitant medications taken within 3 weeks prior to the first administration of visilizumab through Day 60 on the Concomitant Medication page of the patient's CRF.

TABLE 3
Permitted Concomitant Medications for the Treatment of UC
Medication

Steroids (methylprednisolone), IV
6-mercaptopurine
5-ASA (5-aminosalicylic acid)

### 7. STUDY PARAMETERS

### 7.1. Demographics and Baseline Characteristics

The demographic and baseline characteristics of interest include age, sex, race/ethnicity, disease duration and severity, prior therapies, and baseline MTWSI and Mayo System scores.

### 7.2. Safety

Safety variables include adverse events (AE), serious adverse events (SAE), opportunistic infections, malignancies, surgeries, patient clinical status (vital signs and temperature), and laboratory values (complete blood counts including differential and platelet count, serum chemistries, and quantitative EBV testing by PCR).

7.2.1. Adverse Events

Adverse events (AE) were presented in listings. Each AE was classified according to a preferred term and body system using a MedDRA or COSTART thesaurus. The number and proportion of patients reporting AEs were summarized according to body system and preferred term.

7.2.2. Clinical and Laboratory Assessments

The clinical status of each patient was monitored by recording AEs, SAEs, opportunistic infections and malignancies, changes in vital signs, and laboratory analyses.

7.2.3. Special Assessments

To exclude patients with CMV colitis, a biopsy of intestinal mucosa was performed during the screening sigmoidoscopy, within 3 weeks prior to Day 1 (first visilizumab dose). If >1 CMV inclusion body was observed per high-powered field upon pathology examination, the patient would be disqualified from participating in the study.

### 7.3. Pharmacokinetics

The serum concentrations of visilizumab obtained throughout the study were used to analyze the pharmacokinetic (PK) profile of visilizumab over time. Serum samples were collected from each patient prior to visilizumab dosing and at various time points after dosing as specified in Section 4. Standard PK parameters, including the maximum serum concentration (Cₘₐₓ), time of Cₘₐₓ, area under the time-concentration curve (AUC), and serum half-life of visilizumab was determined.

### 7.4. Pharmacodynamics

Pharmacodynamic data included total and peripheral T-cell counts. Blood samples for measuring peripheral T-cell counts (to evaluate T-cell depletion/recovery) were collected from each patient before and after the first dose of visilizumab and at several intervals up to Day 30. If CD3⁺CD4⁺ T-cell counts have not reached ≧200 cells/μL or >50% of patient's baseline value by Day 30, testing continued every 7 days until this T-cell level was reached.

### 7.5. Immunogenicity (Anti-Ab)

Serum samples for the analysis of an antibody response to administered humanized antibody (Anti-Ab) were collected from each patient on the days and time points specified in Section 4. Samples were shipped to PDL for analysis.

### 7.6. Efficacy Parameters

In this study, the MTWSI will be used to measure cross-sectional disease activity in UC patients at baseline and on Day 1 before study drug administration; at 15, 30, 60, and 90 days after; and at 6 months after, study drug administration. The Mayo Scoring System will be used to measure disease activity in UC patients at baseline and on Day 30 only.
Modified Truelove & Witts Severity Index (MTWSI)

The MTWSI is a standardized rating scale used by the treating physician to classify disease severity in UC patients. Disease symptoms are graded using individual scales for diarrhea, nocturnal diarrhea, rectal bleeding, fecal incontinence, abdominal cramping, general well being, need for antidiarrheals, and abdominal tenderness. Each category has its own scale (range 0 to 1-5) (0=normal and higher numbers reflect increasing severity); a maximum total score is 21 points (see Table 4, Modified Truelove and Witts Severity Index). In this study, the MTWSI score for each symptom category (except abdominal cramping) was calculated as a 3-day average, covering the period immediately preceding the current assessment. All averages were rounded to the nearest whole number, including those for symptoms that are scored as Yes (1) or No (2).

Serial changes from baseline were calculated. Response to treatment was defined as an absolute MTWSI score of <10. Remission in these UC patients was defined as an MTWSI score of <3.
Mayo Scoring System for Assessment of Ulcerative Colitis Activity

The Mayo Scoring System is another standardized rating scale used by the treating physician to classify disease severity in UC patients. Disease symptoms are graded using individual scales for stool frequency, rectal bleeding, a physician's global assessment (PGA), and the findings of flexible proctosigmoidoscopy. Each category has its own scale (range 0 to 3) (0=normal; higher numbers reflect increasing severity); a maximum total score is 12 points (see Table 5, Mayo Scoring System for Assessment of Ulcerative Colitis Activity). The symptoms of stool frequency and rectal bleeding were scored as a 3-day average, covering the period immediately preceding the current assessment. All averages were rounded to the nearest whole number. The PGA score acknowledges the other 3 criteria, the patient's daily record of abdominal discomfort and general sense of well-being, and other observations, such as physical findings and the patient's performance status.

A total Mayo UC activity score of 0 to 2 points indicates remission or minimally active disease; a score of 3 to 5 points indicates mildly active disease; a score of 6 to 10 points indicates moderately active disease; and a score of 11 to 12 may indicate moderate or severe disease, depending on the patient's MTWSI score.

### 8. ANALYTICAL METHODS

### 8.1. Overall Assumptions

For this Phase I study, results were summarized by dose level without formal statistical testing of between-group differences. Descriptive statistics and 95% confidence intervals were employed where appropriate.

### 9.2. Demographics

Demographic data (ie, age, sex, and race/ethnicity), disease duration and severity, prior therapies, smoking history, and baseline MTWSI score were summarized by dose level and tabulated by patient.

### 9.3. Safety

### 9.4. Pharmacokinetics

Serum visilizumab concentrations were used to calculate standard PK parameters, including Cₘₐₓ, AUC, clearance, and serum half-life (T_{1/2}). All measurable results were tabulated and presented graphically by patient or by dose group.

### 9.5. Pharmacodynamics

Total and subpopulation T-cell counts were presented in patient listings. Mean peak values of T-cell counts were graphed over time by dose level.

### 9.6. Immunogenicity (Anti-Ab)

Serum samples were shipped to PDL for ELISA analysis of levels of circulating antibodies to administered visilizumab (Anti-Abs). Any detectable antibody response was tabulated by subject, and the frequency of response was tabulated by dose group.

### 9.7. Efficacy Parameters

Changes from baseline over time in the MTWSI and Mayo scores were summarized by dose level. If appropriate, the significance of the within-group changes was statistically assessed with Wilcoxon Signed Rank tests. No formal statistical between-group comparisons were planned.

Response and remission rates at different time points were described by point estimates and exact 95% confidence intervals (CI), as calculated by StatXact-4© (Cytel Software Corporation). If, at the OBD, 21 of the 30 patients respond, this would yield a point estimate of 70% and a 95% CI delimited by 51% and 85%.

### 9.8. Patient Disposition

An accounting of all patients over the course of the study will be reported by dose group. Distribution of patients will be reported by treatment group assignment and investigator, and eligibility status. In addition, the disposition of all patients screened but excluded or unwilling to participate (screen failures) will be reported by principal reason for noninclusion in the study.

A complete description of this protocol is found in PDL's Protocol Number 291-406 (“A Phase I, Dose-Escalation, Pilot Study of Visilizumab in patients With Severe Ulcerative Colitis That is Refractory to Corticosteroids”), Visilizumab (Nuvion®; HuM291), dated Nov. 14, 2001; Amendment A: Sep. 16, 2002; Amendment B: Nov. 27, 2002 (which is herein incorporated by reference in its entirety).

TABLE 4
Modified Truelove and Witts Severity Index
Modified Truelove & Witts Severity Index

SubTotal

*Diarrhea (Total number of bowel movements [BM]/day)

| | | |
|---|---|---|
| 0 = 1-2 BM/day | 3 = 7-9 BM/day | |
| 1 = 3-4 BM/day | 4 = 10+ BM/day | |
| 2 = 5-6 BM/day | | = _{———} |

*Nocturnal Diarrhea/Early AM Awakening for BM

| | | |
|---|---|---|
| 0 = No | 1 = Yes | = _{———} |
| *Bloody Stool | | |
| 0 = None | 2 = ≧50% of BM | |
| 1 = Occasionally | 3 = with every BM | |
| with BM | | = _{———} |

*Fecal Incontinence/Soiling

| | | |
|---|---|---|
| 0 = No | 1 = Yes | = _{———} |

*Abdominal Pain/Cramping

| | | |
|---|---|---|
| 0 = None | 2 = Moderate - Interferes with usual | |
| | activities | |
| 1 = Mild - Aware, | 3 = Severe - Incapacitating | |
| but tolerable | | = _{———} |

*General Well Being

| | | |
|---|---|---|
| 0 = Excellent | 3 = Fair | |
| 1 = Very Good | 4 = Poor | |
| 2 = Good | 5 = Terrible | = _{———} |

*AntiDiarrheals/Narcotics

| | | |
|---|---|---|
| 0 = No | 1 = Yes | = _{———} |

Abdominal Tenderness

| | | |
|---|---|---|
| 0 = None | 2 = Mild to Moderate & Diffuse | |
| 1 = Mild to | 3 = Severe or Rebound Tenderness | |
| Moderate & | | |
| Localized | | = _{———} |

| | |
|---|---|
| Total MTWSI Score → | = _{———} |

*In each indicated symptom category, the MTWSI score is calculated as a 3-day average, covering the period immediately preceding the current assessment. All averages are rounded to the nearest whole number, including those for symptoms that are scored as Yes (1) or No (2).

TABLE 5
Mayo Scoring System for Assessment of Ulcerative Colitis Activity
MAYO Severity Index

| | SubTotal |
|---|---|
| Stool Frequency^{a} (Total number of stools/day) | |
| (3-day average^{b}) | |
| 0 = Normal number of stools for this patient | |
| 1 = 1-2 stools/day more than normal for this patient | |
| 2 = 3-4 stools/day more than normal for this patient | |
| 3 = ≧5 stools/day more than normal for this patient | = _{———} |
| Rectal Bleeding^{c} (3-day average^{b}) | |
| 0 = No blood seen | |
| 1 = Streaks of blood with <50% of stools | |
| 2 = Obvious blood seen with ≧50% of stools | |
| 3 = Blood alone passed | = _{———} |
| Physician's Global Assessment (PGA)^{d} | |
| 0 = Normal | |
| 1 = Mild disease | |
| 2 = Moderate disease | |
| 3 = Severe disease | = _{———} |
| Finding of Flexible Proctosigmoidoscopy | |
| 0 = Normal or inactive disease | |
| 1 = Mild disease (erythema, decreased | |
| vascular pattern, mild friability) | |
| 2 = Moderate disease (marked erythema, absent vascular | |
| pattern, friability, erosions) | |
| 3 = Severe disease (spontaneous bleeding, ulceration) | = _{———} |
| Total MAYO Score → | = _{———} |

^{a}Each patient serves as his or her own control to establish the degree of abnormality of the stool frequency.
^{b}The 3-day average includes the 3-day period immediately preceding the current assessment. All averages are rounded to the nearest whole number.
^{c}The daily bleeding score represents the most severe day of bleeding.
^{d}The PGA score acknowledges the other 3 criteria, the patient's daily record of abdominal discomfort and general sense of well-being, and other observations, such as physical findings and the patient's performance status.

### EXAMPLE 3

This example describes the results of humanized anti-CD3 monoclonal antibody, Visilizumab, for treatment of severe steroid-refractory ulcerative colitis in the first 10 patients.

In severe, steroid-resistant ulcerative colitis (UC), therapeutic approaches have been used to targeted T-cells to control inflammation. For example, cyclosporine is efficacious in this population over the short-term, but side effects limit its use. Humanized anti-CD3 monoclonal antibody, visilizumab (Protein Design Labs, Inc., Fremont, Calif.), which induces preferential apoptosis of activated T-cells in vitro, provides therapeutic benefit in UC.

Of the ten patients treated, eight were given a visilizumab dose of 15 μg/Kg and two were given a visilizumab dose of 10 μg/Kg. Of the ten, six were male and four were female. The age ranged from 33 years old to 70 years old. The median age was 46 years old. Of the disease extent, four had left sided UC and six had pancolitis UC. The MTWSI score at enrollment was 11-14, with the median score being 13.25. The EBV whole blood viral DNA copies was <80 mL. The drug regimen was methylprednisolone (MP) for ten patients, 5-aminosalicylic acid (5-ASA) for five patients, and azathioprine for one patient. The hemocrit value had a range of 28.5 to 45.3%, with a mean of 36.3%. The albumin content had a range of 2.4-3.5 g/dL, with a mean of 3.0 g/dL. The ESR value was 5-54 mm/hr, with a mean of 26 mm/hr.

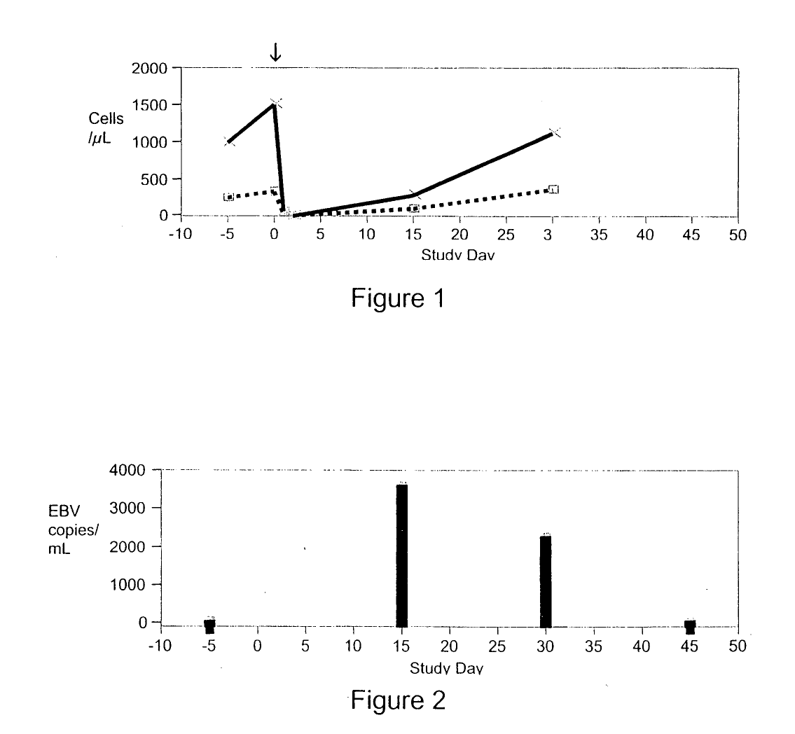

The safety assessments of the phase I UC study, included observation for acute toxicities and intermediate effects. For acute toxicities, on day 1 of treatment, eight of the ten patients had mild to moderate Cytokine Release Syndrome (CRS). CRE is characterized by fatigue, nausea, chills, headache, arthralgia, fever, emesis, dehydration, dizziness, and diaphoresis. These symptoms are transient and typically last 1-2 hours after infusion. On day 2, five of the ten patients had CRS where the symptoms were reduced in intensity and frequency, 1 DLT. Intermediate effects include T-cells reaching nadir levels hours after each dose. For six of eight patients, T-cells then recover to >200 CD4/μL in 2-6 weeks (see FIG. 1). Two patients experienced delayed recovery. Both eventually recovered but the specific day of recovery could not be determined due to the long inter-assessment periods. For most patients the EBV whole blood viral DNA copies fluctuated inversely to the T-cell counts. Six of eight patients experienced transient rises (range of 184-3640; mean of 1468). For three patients the EBV whole blood viral DNA copies were undetectable by day 30, and that for the remaining three became undetectable by day 60 (see FIG. 2).

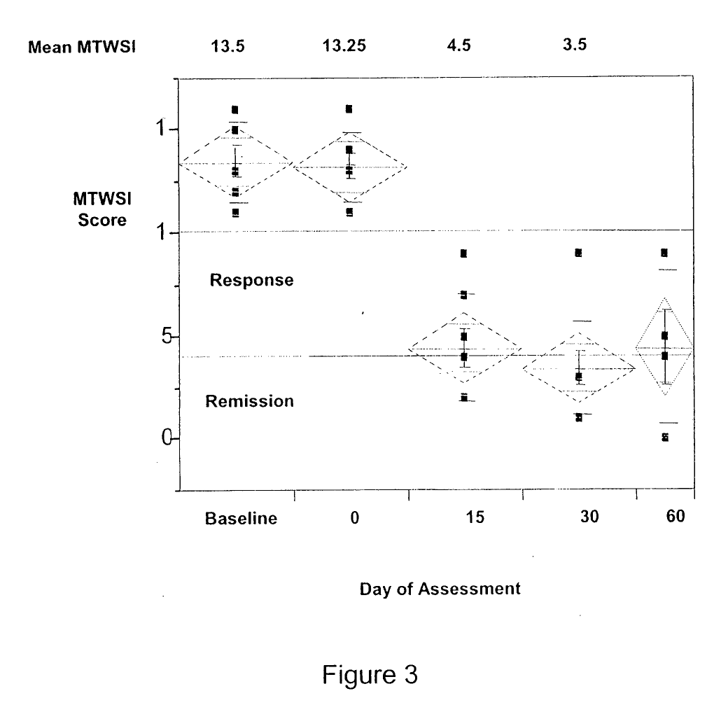

Efficacy of the treatment was measured by determining the MTWSI score for each patient that received 15 μg/Kg day 1 and day 2. The results of the clinical response of eight patients on 15 μg/Kg day 1 and day 2 are complied in FIG. 3. The baseline mean MTWSI score is 13.5. The mean MTWSI score at day 0 is 13.25. On day 15, the mean MTWSI score is 4.5 (9.0 less than the baseline mean MTWSI score). On day 30, the mean MTWSI score is 3.5 (10.0 less than the baseline mean MTWSI score). A MTWSI score of <4 at 30 days is considered a “remission”. “Remission” indicates a decrease in the MTWSI to less than or equal to 4 sustained for 60 days. Seven of the eight patients reached this endpoint. A MTWSI score of <10 at 30 days is considered a clinical “response”. “Response indicates a decrease in the MTWSI of at least 2 points to below a value of 10 sustained for at least 30 days. Seven of the eight patients reached this endpoint. Each of the eight patients reached this endpoint. The MTWSI score at 30 days showed a 74% mean reduction from baseline P<0.0039.

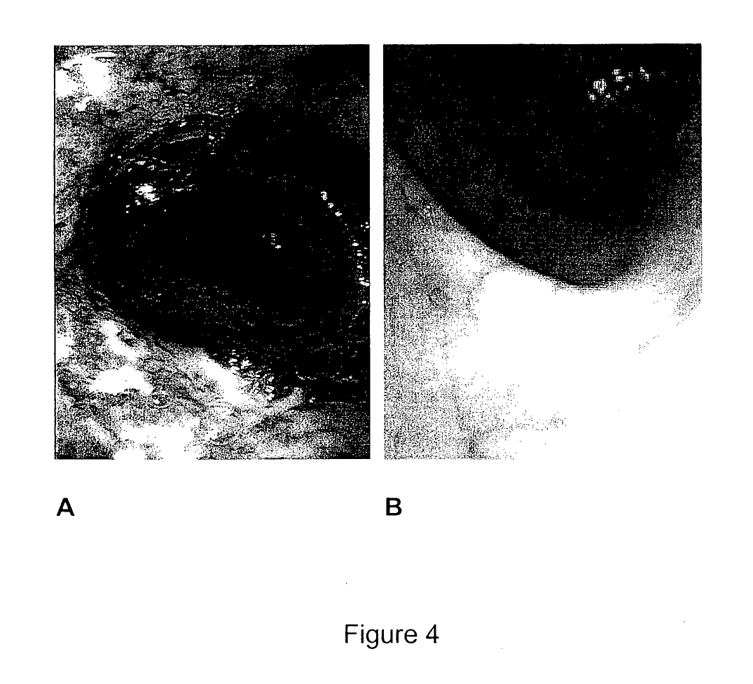

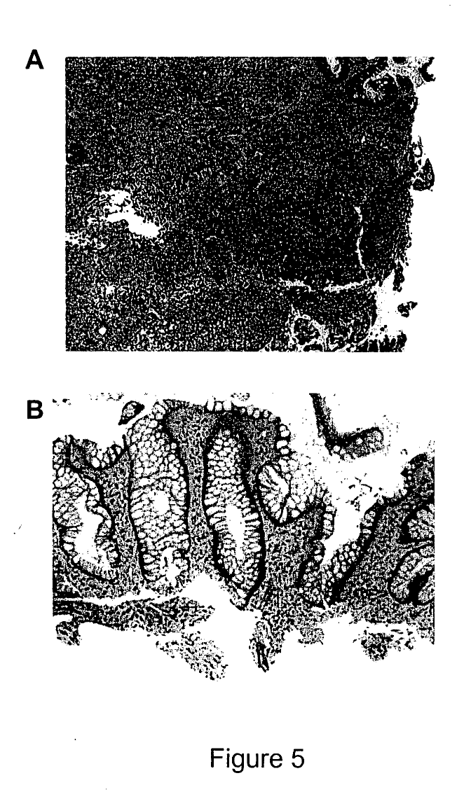

Regarding endoscopic examination, eight of eight patients showed endoscopic improvement at day 30. Seven of eight patients had a severe condition at the time of treatment, and six of eight patients had a mild or normal condition at 30 days. FIG. 4 shows the endoscopic response after 30 days of treatment compared to the pre treatment. The pre treatment colon afflicted with UC has spontaneous bleeding and ulceration. FIG. 5 shows the histologic response after 30 days of treatment compared to the pre treatment. The pre treatment colonic muscosa, taken from a colon afflicted with UC, shows neutrophils in the lamina propria and increased lymphocyte and plasma cells. Efficacy of the treatment was measured by determining the MTWSI score for two patients that received 10 μg/Kg day 1 and day 2. Two patients were recorded with “responses” at day 15, and one had recorded “response” at day 30. The follow-up observations indicated that the median duration of response was 7 months (with a range of 2-11 months). Of the eight patients who received the 15 μg/Kg dosage: six of the eight patients in remission were off steroids 5-1 months post therapy, and two of eight patients initially responded but later had a colectomy on day 62 and 100.

Of eleven patients undergoing the visilizumab therapy, one discharged from the hospital two days from the first day of visilizumab infusion, five patients discharged from the hospital three days from the first day of visilizumab infusion, three patients discharged from the hospital four days from the first day of visilizumab infusion, and two patients discharged from the hospital five days from the first day of visilizumab infusion. There was a mean of 3.5 days and a median of three days to hospital discharge from the first day of visilizumab infusion. These results indicate that patients treated with 10 or 15 μg/Kg day 1 and day 2 can be discharged from hospital in a relatively short period of time. These results are compared with stays of 7-14 days following cyclosporine A treatment or colectomy. The speed of response is remarkable both its impact in reducing hospital costs and in the potent activity against very active disease. Clearly, inpatients whose disease is uncontrollable and severe, a rapid response is necessary to prevent colectomy.

At present, based on the patients evaluated, there is no evidence that transient increases in patient EBV levels subsequent to treatment with visilizumab are clinical significant. There is only mild to moderate cytokine release. The transient T-cell decreases in peripheral blood. Recovery 2-6 weeks to baseline. The EBV titers elevate transiently and return to undetectable levels priori to T-cell recovery to baseline levels. Significant early clinical response noted in very refractory patient group who are typical surgical candidates.

### EXAMPLE 4

This example describes the results of humanized anti-CD3 monoclonal antibody, visilizumab, for treatment of severe steroid-refractory ulcerative colitis. The following results incorporate the results reported in Example 3.

In severe, steroid-resistant ulcerative colitis (UC), therapeutic approaches have been used to targeted T-cells to control inflammation. For example, cyclosporine is efficacious in this population over the short-term, but side effects limit its use. Humanized anti-CD3 monoclonal antibody, visilizumab (Protein Design Labs, Inc., Fremont, Calif.), which induces preferential apoptosis of activated T-cells in vitro, provides therapeutic benefit in UC.

These preliminary results are from a multicenter, phase I study of visilizumab in patients with severe UC whose disease has not responded to a minimum of 5 days of intravenous (IV) corticosteroids with undetectable levels of EBV. Twenty-three patients received an IV infusion of visilizumab on study days 1 and 2. The first 8 received a dose of 15 μg/Kg, the next 18, 10 μg /Kg. The patients have been followed for a median of 80 days (8-516) from treatment.

The 23 patients had a median baseline MTWSI of 13.6 (11-20). Three patients failed to have an initial response sustained at least 30 days. A fourth patient had a colectomy on day 102. The 19 responding patients have continued to maintain clinical improvement for up to 16 months following treatment. One patient's disease flared at one year post treatment. Transient (1-4 week) decreases in T-lymphocyte counts from peripheral blood were observed. 1/11 (10 μg/Kg) and 2/8 (15 μg/Kg) had less than 200 CD3+4+cells/μL persisting on day 30. All patients recovered by day 60. Mild to moderate cytokine release symptoms (nausea, vomiting, chills, arthralgias) were observed in 12/16 patients. These symptoms were transient, resolving within 1-2 hours and occurred predominantly on day 1. Thirteen of 17 patients had transient Epstein-Barr copy titers in whole-blood detected by PCR (median 566 (153-190000) on day 15) that were not associated with clinical symptoms. All patients returned to undetectable levels by day 60. There have been no documented infectious complications.

This preliminary analysis of an open-label phase I study of visilizumab in patients with severe UC has demonstrated potential tolerability and clinical activity at a very low dose. This approach provides an important therapeutic option for this patient population.

### EXAMPLE 5

### Multiple Dosage Trial of Visilizumab on Patients with Intravenous Steroid-Refractory Ulcerative Colitis (IVSR-UC)

The previous trials were started at a dosage of 15 μg/kg with an intent to increase dosage if side effects were not observed at the initial dosage. 15 μg/kg is already a low dosage compared with most approved therapeutic antibodies, which are usually administered in mg/kg quantities. In fact, however, the dosage of 15 μg/kg gave a virtually 100% response rate but also significant side effects in terms of cytokine release syndromes and delayed T-cell recovery. Thus, a further trial was performed to assess a range of lower dosages of visilizumab when administered to patients with severe ulcerative colitis refractory to IV steroids (IVSR-UC). Dosages tested were between 5 and 12.5 μg/kg. The terminology used in this example has the same meaning as in previous examples.

Patients enrolled in the trial were stratified according to presence or absence of EBV DNA into two categories: 1. EBV minus patients were randomized equally to 4 dose levels (5, 7.5, 10, and 12.5 μg/kg); and 2. EBV plus (greater than 5000 copies/mL) were initially enrolled into the lowest dose level and dose escalated if they tolerated treatment at the lower dose level. An optimal clinical dose (OCD) was determined and additional patients were enrolled at the OCD. The protocol allowed for enrollment of up to 144 patients, including up to 21 patients in each of the 4 dose cohorts in stage 1 and up to 60 more at OCD in stage 2.

A Visilizumab bolus intravenous (IV) dose was given on days 1 and 2 only, the dosages ranged from 5μg/kg to 12.5 μg/kg. A standardized pre-treatment regimen of IV cortocosteroids, diphenhydramine, ondansetron, and prehydration was administered 1 hour before visilizumab infusion.

Inclusion and Exclusion Criteria of the Study

The major inclusion criteria included 1. diagnosis of ulcerative colitis verified by colonoscopy or barium enema performed within 36 months prior to the study entry; 2. Active disease documented by MTWSI Index score of 11-21 despite treatment course of IV steroids for a minimum of 5 days; and 3. Tested negative for C. Difficile within 10 days. The exclusion criteria included the following: 1. ulcerative colitis requiring immediate intervention, history of total proctocolectomy or subtotal colectomy with ileorectal anastomosis, white blood cells count (WBC) less than 2.5×10³ μL, platelet less than 150×10³/μL, or hemoglobin less than 8 G/dL, patients with serious infections, particularly those of viral etiology, including opportunistic infections within the past year, patients who have received live vaccine within 6 weeks, patients with a history of thrombophlebitis or pulmonary embolus, significant organ dysfunction including cardiac, renal, liver, CNS, pulmonary, vascular, GI, endocrine or metabolic, patients with a history of LPD or malignancy other than non-melanoma skin cancer or carcinoma in situ of the cervix that has been adequately treated, HIV, HCV or HBV seropositivity, EBV DNA greater than 5,000 copies/mL in Stage 1, treatment with antibody or investigational agent within 60 days, treatment with CsA or Tacrilimus within 3 months, a history of seizures, history of chronic and current treatment with anticonvulsant medications, no documentation of therapeutic blood levels of anticonvulsant medication within 7 days, or pregnant women or nursing mother.

Results

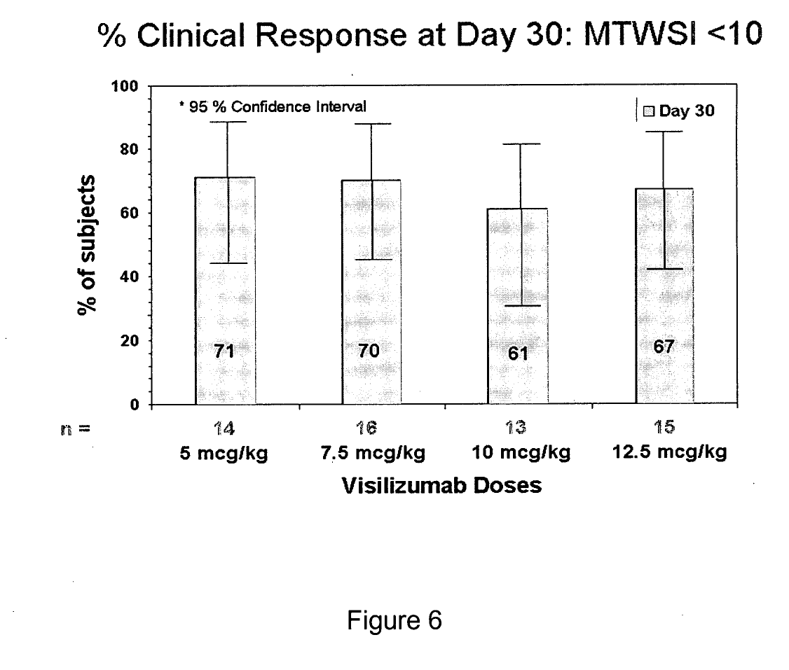

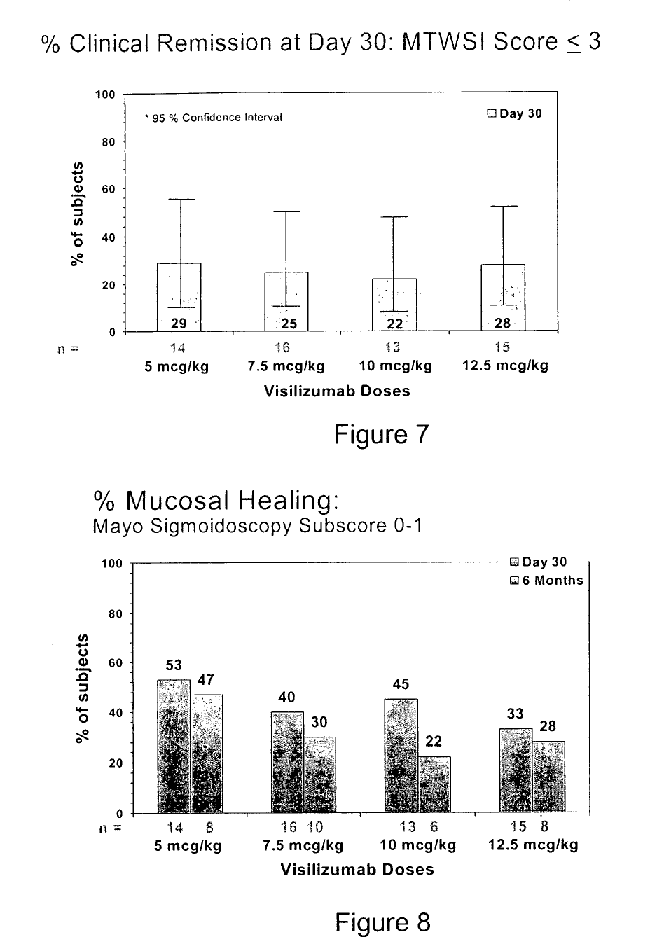

FIG. 6 shows the % clinical response at day 30 (MTWSI less than 10) was 71% at 5 μg/kg, 70% at 7.5, 61% at 10, and 67% at 12.5 μg/kg. FIG. 7 shows that the clinical remission rate (MTWSI≦3) was 29%, 25%, 22%, and 28% respectively. Remission requires at least three point improvement in the Mayo score from baseline. When tested at 60 and 90 days post-treatment, the remission rate was the same or only slightly reduced relative to 30 days.

FIG. 8 shows the mucosal healing at day 30 was significant for each dosage at day 30 and 6 months with better healing using the lower dose of 5 μg/kg. The healing did not improve with time. Table 6 shows a summary of the efficacy endpoints. The table compares values obtained as evaluated using two indices, the MTWSI and the Mayo scores. MTWSI is the Modified Truelove Watts Index and is a measure of disease activity using a variety of parameters. The Mayo scores range from 0 to 12 with higher scores indicating more severe disease.

TABLE 6
Summary of Efficacy Endpoints

| | 5 μg/kg | 7.5 μg/kg | 10 μg/kg | 12.5 μg/kg |
|---|---|---|---|---|
| | (n = 17) | (n = 20) | (n = 18) | (n = 18) |
| MTWSI | | | | |
| % Response day 30 | 71% | 70% | 61% | 67% |
| % Remission day 30 | 29% | 25% | 22% | 28% |
| Mayo | | | | |
| % Response day 30 | 71% | 65% | 50% | 61% |
| % Remission day 30 | 29% | 15% | 6% | 11% |
| % mucosal healing | 53% | 40% | 45% | 33% |
| day 30 | | | | |

Adverse Effects

Adverse effects were identified and measured using a designation of from mild to severe. Although adverse effects were seen in greater than or equal to 10% of subjects, they were rarely severe or life threatening. The details of the adverse effects were as follows. Adverse events associated with cytokine release symptoms (CRS) included chills, headache, pyrexia, transient LFT elevations, fatigue, arthralgia, back pain, abdominal pain, nausea, dizziness and tachycardia. There was a dose-related increase in moderate/severe CRS adverse events as shown below.

TABLE 7
Dose dependence of adverse events

| | 5 mcg/kg | 7.5 mcg/kg | 10 mcg/kg | 12.5 mcg/kg |
|---|---|---|---|---|
| | (n = 17) | (n = 20) | (n = 18) | (n = 18) |
| Patients who | 17 (100%) | 19 (95%) | 17 (94%) | 16 (89%) |
| reported | | | | |
| CRS | | | | |
| Grade 1 | 16 (94%) | 14 (70%) | 11 (61%) | 10 (56%) |
| (Mild) | | | | |
| Grade 2 | 10 (59%) | 13 (65%) | 14 (78%) | 13 (72%) |
| (Moderate) | | | | |
| Grade 3 | 0 (0%) | 7 (35%) | 4 (22%) | 3 (17%) |
| (Severe) | | | | |
| Grade 4 | 0 (0%) | 0 (0%) | 1 *(6%) | 0 (0%) |
| (Life | | | | |
| Threatening) | | | | |

Adverse events not associated with CRS included dyspepsia, hemorrhoids, and ulcerative colitis. Most of the adverse events were mild to moderate with some severe events and only one could be considered life threatening (atrial fibrillation on Day 2, recovered by the following day with medication). SAE's reported included Atrial fibrillation, anemia, hemorrhoids, abdominal abscesses, gastroenteritis, esophageal candidiasis, CMV test positive, psychotic disorder, and hypotension. Infectious disease adverse events included nasopharyngitis/sinusitis, upper respiratory tract infection, herpes simplex, herpes zoster ophthalmic, candida, Urinary tract infection (UTI), bacterial infection, gastroenteritis, C. Difficile, onychomycosis, otitis media, pneumonia, sweating fever, abdominal abscess. Only 3 cases were classified as grade 3 (esophageal candidiasis, gastroenteritis, and abdominal abscess).

Dose Limiting Toxicities

The dose-limiting toxicities were defined as one of the following: 1. CD4+ T cells not recovered by Day 60 to at least 200 cells/μL or if CD4+levels were less than 200 cells/μL at baseline, return to baseline level; 2. EBV DNA less than 400 copies/mL by day 60, or if pretreatment EBV DNA greater than 400 copies/mL, then a return to within 1/2 log greater than the higher of 2 pretreatment levels; or 3. Any Grade 4 toxicity (including infusion reactions) or Grade 3 infusion-related toxicities observed beyond 24 hours after the second infusion. The number and type of dose limiting toxicities is shown in Table 8.

TABLE 8
Dose Limiting Toxicities

| Dose | | CD4+ T cell | EBV | |
|---|---|---|---|---|
| μg/kg (N) | Drug Related Severe AEs | recovery | recovery | Total |
| 5 (17) | 2 (abdominal abscess and | 0 | 0 | 2 |
| | hypotension) | | | |
| 7.5 (20) | 1 (CMV +) | 1 | 2 | 4 |
| 10 (18) | 2 (atrial fibrillation and | 2 | 3 | 7 |
| | esophageal | | | |
| | candidiasis) | | | |
| 12.5 (18) | 0 | 4 | 3 | 7 |
| Total | 5 | 7 | 8 | 20 |

Summary

In summary, the Visilizumab at 5 μg/kg×2 doses equally effective in treating ulcerative colitis as higher doses up to 15 μg/kg. However, side effects, particularly cytokine release symptoms and delayed T-cell recovery were dose were dose dependent between 5 μg/kg and 15 μg/kg. These results suggest that a concentration of about 5 μg/kg provides an optimal balance between efficacy and side effects in a regime in which doses are administered per episode of UC. Visilizumab could also be used at higher concentration because in general the adverse effects were transient and/or could be resolved completely. However, the present results indicate there is no therapeutic benefit in using larger dosages. Visilizumab can be used for EBV+ patients and showed a similar outcome for both EBV+ and EBV− patients. Visilizumab did not result in adverse events in EBV+ patients that would preclude treatment, especially at the lowest optimal dose.

### EXAMPLE 6

### Treatment of Crohn's Disease Refractory to Infliximab Treatment

Crohn's disease is an inflammation primarily of the small intestine that can also affect the esophagus, stomach, colon, and other organs and tissues. The prevalence of the disease in the United States is estimated at approximately 100 cases per 100,000 individuals. Symptoms of Crohn's disease include fever, abdominal pain, diarrhea, weight loss, and generalized fatigability. Recurrence is common and unpredictable, and severely affects the patient's quality of life. Current drug treatments for Crohn's disease include anti-inflammatory and immunosuppressive agents including antagonists of TNF-alpha such as Remicade® (infliximab). However, the response to these agents frequently decreases over time and the disease often becomes chronic, leading in many cases to repeated surgical intervention (Harrison's Principles of Internal Medicine, 14.sup.th Edition, pp. 1643 (1998)).

Visilizumab was tested for treatment of Crohn's disease. The study was performed using 10 μg/kg administered in an IV bolus on each of 2 consecutive days. This dose, which is much lower than the dose of most approved antibodies, was selected because of dose limiting toxicities (particularly delayed recovery of T-cells) observed in some patients at the 15 μg/kg dose in previous trials for treatment of ulcerative colitis.

The clinical activity of visilizumab in patients with moderate to severe inflammatory, nonstricturing, nonpenetrating forms (luminal) of Crohn's disease was evaluated. Many of the patients enrolled in the study were refractory to treatment with infliximab (an approved anti-TNF-α antibody). The severity of Crohn's disease can be measured using the levels of C-reactive protein (CRP) and Crohn's disease activity index scores (a quantitative measurement of the severity of active disease using symptom scores that are monitored over 1 week, known as CDAI). A reduced CRP indicates reduced disease and/or remission. The CDAI scores are based on a number of criteria. A CDAI score of less than 150 indicates a better prognosis than higher scores. Subjects with a CDAI>250, a CRP>4 mg/L, and endoscopic evidence of active disease were administered intravenous bolus injections of visilizumab 10 μg/kg on 2 consecutive days (Days 0 and 1).

Clinical response was defined as a decrease in the CDAI of >100 points below the subjects' baseline value, and remission as a CDAI<150 at day 59. Biopsies from a subset of 6 subjects were evaluated by immunohistochemistry at pre-therapy and day 59. Seventeen of 18 enrolled subjects received both doses (one subject withdrew after the first dose); 13 treated subjects were followed through to day 89. The median baseline CDAI was 396 (271-516), and the median baseline CRP was 28 mg/L (2-70). Nine subjects were oral or IV steroid-resistant, 6 were oral or IV steroid-dependent, and 3 were steroid-independent. Seventeen subjects had received prior infliximab (IFX); 5 were primary IFX nonresponders, 2 had had intolerable infusion reactions, and 10 had discontinued IFX due to loss of activity.

Inclusion and Exclusion Criteria of the Study

The major inclusion criteria included 18 to 70 years of age, moderate to severe inflammatory, nonstricturing, nonpenetrating forms of Crohn's disease, Crohn's disease activity Index (CDAI) of greater than or equal to 250, CRP of greater than 4 mg/L, endoscopic evidence of moderate to severe active inflammatory disease, tested negative for C. difficile within 3 weeks prior to treatment with study drug, and EBV DNA titers up to 30,000 copies/mL. Exclusion criteria included, any of the following hematologic abnormalities: WBC less than 2500/mm3, platelets less than 150,000/mm3, hemoglobin less than 10 g/dL; serious infections or serologic evidence of infection with HIV or HBV or HCV; had an increased dose in corticosteroid medication 2 weeks prior to receiving a study drug or is receiving IV steroids or is receiving a daily dose of greater than 40 mg prednisone, less than 9 mg budasonide, or equivalent; received any monoclonal antibodies including infliximab or investigational agents or biologics within 3 months prior to receiving study drug; receiving cyclosporine or tacrolimus (FK506) within 4 weeks of receiving study drug; or a history of tuberculosis.

Results

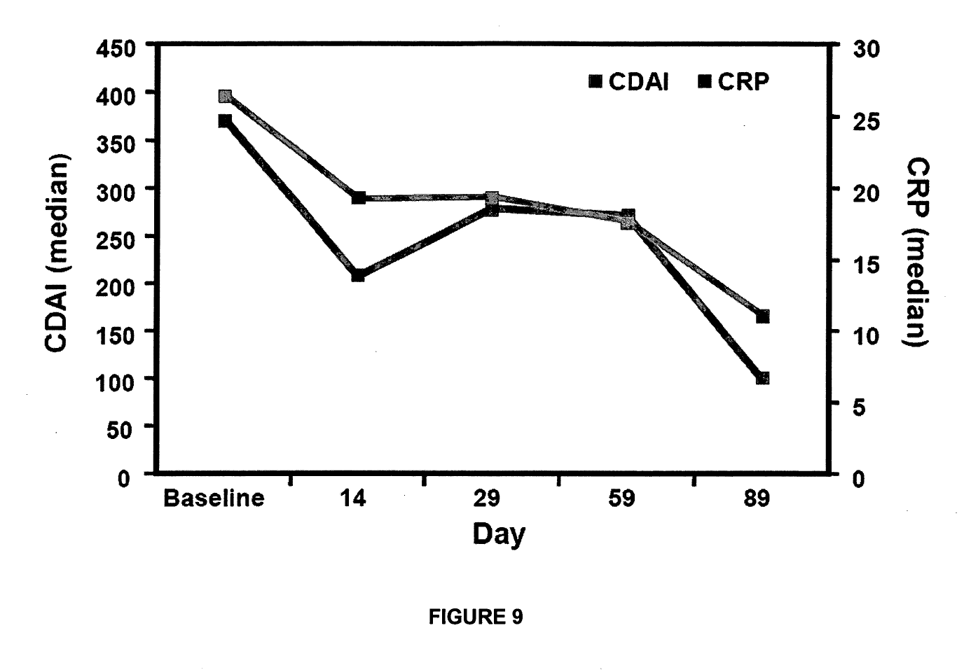

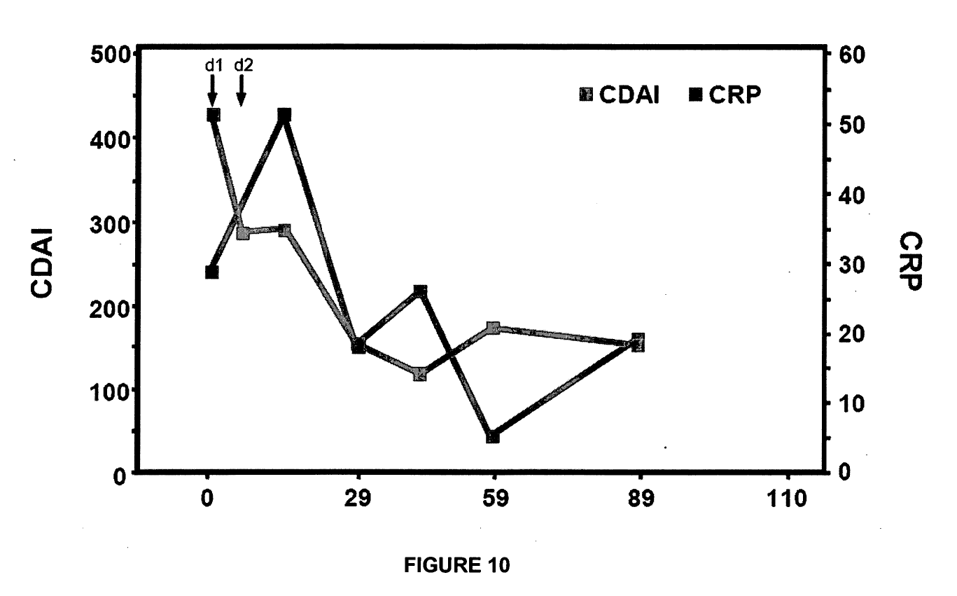

By Day 29, responses were seen in 11 of 18 subjects, clinical remission in 5 of the 18. More than half (13/18) of the subjects had a clinical response to visilizumab at Day 59, and 5 were responders at 6 months. Remission by Day 59 was achieved in 6 of the 18 subjects. The median D59 CDAI was 266 (69-570). On average, in visilizumab responders, the CRP decreased by ≧50% by or before Day 15. By Day 59, all 6 subjects for whom paired biopsies were available showed evidence of mild to moderate histological improvement. FIG. 9 shows the course of CRP and CDAI in patients. FIG. 10 shows the course in responding patients.

Adverse Effects

Mild to moderate cytokine release syndrome (CRS) occurred in the majority of subjects. The adverse event (AE) profile, including their severity, incidence, and drug relatedness were similar to that previously seen in IVSR-UC subjects who had received an equivalent dose of visilizumab (10 μg/kg×2). No lymphoproliferative, malignant or life-threatening adverse events were reported. Infectious disease adverse effects were all mile to moderate and included Clostridial infection, Herpes Simplex reactivation, influenza, nasopharyngitis, and rhinitis.

Summary

Two 10 μg/kg visilizumab doses administered as IV bolus injections on consecutive days were tolerated and produced a rapid and sustained improvement in 11 of the 18 subjects tested with moderate-to-severe, refractory Crohn's disease. These results indicate that the same or similar regime of visilizumab is effective in treating Crohn's disease as for ulcerative colitis. Beneficial effects of treatment include significant reduction in CDAI, and CRP and improved mucosal healing.

Although the invention has been described with reference to the presently preferred embodiments, it should be understood that various modifications may be made without departing from the spirit of the invention. All publications, patents, patent applications, and web sites are herein incorporated by reference in their entirety to the same extent as if each individual patent, patent application, or web site was specifically and individually indicated to be incorporated by reference in its entirety. Unless otherwise apparent from the context any step, feature, aspect, element or embodiment of the invention can be used in combination with any other

1. A method of treating Crohn's disease or ulcerative colitis comprising administering to a patient having the disease an anti-CD3 antibody at a dose of 1-15 μg/kg patient body weight.

2. The method of claim 1, wherein the dose is 1-10 μg/kg.

3. The method of claim 1, wherein the dose is 1-5, 5-10, 5-15 or 10-15 μg/kg.

4. The method of claim 1, wherein the dose is 4-6 μg/kg.

5. The method of claim 1, wherein the dose is 5 μg/kg.

6. The method of claim 1, wherein the dose is 8-12 μg/kg.

7. The method of claim 1, wherein the dose is 10 μg/kg.

9. The method of claim 1, wherein the dose is 2.5-5, 2.5-7.5 or 2.5-10 μg/kg.

10. The method of claim 1, wherein the dose is 2.5 μg/kg patient body weight.

11. The method of any of claim 1, wherein a single dose is administered per relapse of the disease.

12. The method of any of claim 1, wherein two and only two doses are administered per relapse of the disease.

13. The method of any one of claim 1, wherein the two doses are administered on consecutive days.

14. The method of claim 1, wherein the patient's disease is refractory to treatment with an antibody to TNF-alpha.

15. The method of claim 1, wherein the patient's disease is refractory to treatment with intravenous corticosteroids.

16. The method of claim 1, wherein the patient has Crohn's disease and the disease is refractory to treatment with an antibody to TNF-alpha.

17. The method of claim 13, wherein the patient has luminal Crohn's disease.

18. The method of claim 1, further comprising administering a corticosteroid before administration of the anti-CD3 antibody.

19. The method according to claim 1, wherein the antibody is a humanized antibody.

20. The method according to claim 16, wherein the humanized antibody is a humanized M291 antibody.

21. The method according to claim 17, wherein the humanized M291 antibody is visilizumab.

22. The method according to claim 1, wherein the antibody binds to the same epitope as visilizumab.

23. The method according to claim 1, wherein said antibody has an amino acid sequence that is at least 80% identical to the amino acid sequence of visilizumab.

24. The method according to claim 14, wherein said antibody has CDR regions that have amino acid sequences that are identical to the amino acid sequences of the CDR regions of visilizumab.

25. The method of claim 1, wherein the antibody has the same constant region as visilizumab.

26. The method of claim 1, wherein the patient has an Epstein Barr viral titer of greater than 5000 copies per ml before administering the antibody.

27. The method of claim 1, wherein the antibody is administered intraveneously or subcutaneously.
